SEQUENCE LISTING (37 sequences)

SEQ ID NO: 1 - protein, 5 aa, Artificial Sequence
  antibody sequence
RSAMG

SEQ ID NO: 2 - protein, 17 aa, Artificial Sequence
  antibody sequence
AIGTRGGSTKYADSVKG

SEQ ID NO: 3 - protein, 17 aa, Artificial Sequence
  antibody sequence
RGPGYPSAAIFQDEYHY

SEQ ID NO: 4 - protein, 5 aa, Artificial Sequence
  antibody sequence
SDTMG

SEQ ID NO: 5 - protein, 16 aa, Artificial Sequence
  antibody sequence
SISSRGVREYADSVKG

SEQ ID NO: 6 - protein, 13 aa, Artificial Sequence
  antibody sequence
GALGLPGYRPYNN

SEQ ID NO: 7 - protein, 5 aa, Artificial Sequence
  antibody sequence
NYAMG

SEQ ID NO: 8 - protein, 17 aa, Artificial Sequence
  antibody sequence
AISWSGGSTSYADSVKG

SEQ ID NO: 9 - protein, 21 aa, Artificial Sequence
  antibody sequence
QFSGADYGFGRLGIRGYEYDY

SEQ ID NO: 10 - protein, 5 aa, Artificial Sequence
  antibody sequence
NYGMG

SEQ ID NO: 11 - protein, 17 aa, Artificial Sequence
  antibody sequence
GISWSGGSTDYADSVKG

SEQ ID NO: 12 - protein, 17 aa, Artificial Sequence
  antibody sequence
VFSGAETAYYPSDDYDY

SEQ ID NO: 13 - protein, 126 aa, Artificial Sequence
  antibody sequence
QVQLQESGGGLVQAGGSLRLSCAASGRTFGRSAMGWFRQAPGKEREFVAAIGTRGGSTKYADSVKGRFTISTDNASNTVYLQMDSLKPEDTAVYRCAVRGPGYPSAAIFQDEYHYWGQGTQVTVSS

SEQ ID NO: 14 - protein, 121 aa, Artificial Sequence
  antibody sequence
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSS

SEQ ID NO: 15 - protein, 156 aa, Artificial Sequence
  antibody sequence
QVQLQESGGGLVQAGGSLRLSCAASGRTFGRSAMGWFRQAPGKEREFVAAIGTRGGSTKYADSVKGRFTISTDNASNTVYLQMDSLKPEDTAVYRCAVRGPGYPSAAIFQDEYHYWGQGTQVTVSSGLEGHSDHMEQKLISEEDLNRISDHHHHHH

SEQ ID NO: 16 - protein, 151 aa, Artificial Sequence
  antibody sequence
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSGLEGHSDHMEQKLISEEDLNRISDHHHHHH

SEQ ID NO: 17 - protein, 130 aa, Artificial Sequence
  antibody sequence
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSS

SEQ ID NO: 18 - protein, 126 aa, Artificial Sequence
  antibody sequence
EVQLVESGGGLVQAGGSLRLSCAASGRPFSNYGMGWFRQAPGKKREFVAGISWSGGSTDYADSVKGRFTISRDNAKNTVYLQMNSLKPEDTAVYYCAAVFSGAETAYYPSDDYDYWGQGTQVTVSS

SEQ ID NO: 19 - protein, 291 aa, Artificial Sequence
  antibody sequence
QVQLQESGGGLVQAGGSLRLSCAASGRTFGRSAMGWFRQAPGKEREFVAAIGTRGGSTKYADSVKGRFTISTDNASNTVYLQMDSLKPEDTAVYRCAVRGPGYPSAAIFQDEYHYWGQGTQVTVSSGGGGSEVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSSGLEGHSDHMEQKLISEEDLNRISDHHHHHH

SEQ ID NO: 20 - protein, 286 aa, Artificial Sequence
  antibody sequence
EVQLQESGGGLVQAGGSLRLSCVTSGSAFSSDTMGWFRQAPGKQRELVASISSRGVREYADSVKGRFTISRDNAKNTVYLQMNSLQPEDTAVYYCNRGALGLPGYRPYNNWGQGTQVTVSSGGGGSEVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSSGLEGHSDHMEQKLISEEDLNRISDHHHHHH

SEQ ID NO: 21 - protein, 5 aa, Artificial Sequence
  Linker sequence
GGGGS

SEQ ID NO: 22 - protein, 156 aa, Artificial Sequence
  antibody sequence
QVQLQESGGGLVQAGGSLRLSCAASGRTFGRSAMGWFRQAPGKEREFVAAIGTRGGSTKYADSVKGRFTISTDNAKNTVYLQMDSLKPEDTAVYRCAVRGPGYPSAAIFQDEYHYWGQGTQVTVSSGLEGHSDHMEQKLISEEDLNRISDHHHHHH

SEQ ID NO: 23 - protein, 261 aa, Artificial Sequence
  antibody sequence
QVQLQESGGGLVQAGGSLRLSCAASGRTFGRSAMGWFRQAPGKEREFVAAIGTRGGSTKYADSVKGRFTISTDNAKNTVYLQMDSLKPEDTAVYRCAVRGPGYPSAAIFQDEYHYWGQGTQVTVSSGGGGSEVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSS

SEQ ID NO: 24 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 25 - protein, 130 aa, Artificial Sequence
  antibody sequence
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADYGFGRLGIRGYEYDYWGQGTQVTVSS

SEQ ID NO: 26 - protein, 130 aa, Artificial Sequence
  antibody sequence
EVQLLESGGGLVQPGGSLRLSCAASGRPFSNYAMSWFRQAPGKEREFVSAISWSGGSTSYADSVKGRFTISRDNSKNTLYLQMNSLRAEDTAVYYCAAQFSGADYGFGRLGIRGYEYDYWGQGTLVTVSS

SEQ ID NO: 27 - protein, 130 aa, Artificial Sequence
  antibody sequence
EVQLLESGGGSVQPGGSLRLSCAASGRPFSNYAMSWFRQAPGKGLEFVSAISWSGGSTSYADSVKGRFTISRDNSKNTLYLQMNSLRAEDTAVYYCAAQFSGADYGFGRLGIRGYEYDYWGQGTLVTVSS

SEQ ID NO: 28 - protein, 130 aa, Artificial Sequence
  antibody sequence
EVQLLESGGGLVQPGGSLRLSCAASGRPFSNYAMGWFRQAPGKEREFVAAISWSGGSTSYADSVKGRFTISRDNSKNTVYLQMNSLRAEDTAVYYCAAQFSGADYGFGRLGIRGYEYDYWGQGTLVTVSS

SEQ ID NO: 29 - protein, 130 aa, Artificial Sequence
  antibody sequence
EVQLLESGGGSVQPGGSLRLSCAASGRPFSNYAMGWFRQAPGKEREFVAAISWSGGSTSYADSVKGRFTISRDNSKNTLYLQMNSLRAEDTAVYYCAAQFSGADYGFGRLGIRGYEYDYWGQGTLVTVSS

SEQ ID NO: 30 - protein, 130 aa, Artificial Sequence
  antibody sequence
EVQLLESGGGLVQPGGSLRLSCAASGRPFSNYAMGWFRQAPGKEREFVSAISWSGGSTSYADSVKGRFTISRDNSKNTVYLQMNSLRAEDTAVYYCAAQFSGADYGFGRLGIRGYEYDYWGQGTLVTVSS

SEQ ID NO: 31 - protein, 130 aa, Artificial Sequence
  antibody sequence
EVQLLESGGGSVQPGGSLRLSCAASGRPFSNYAMGWFRQAPGKEREFVSAISWSGGSTSYADSVKGRFTISRDNAKNTVYLQMNSLRAEDTAVYYCAAQFSGADYGFGRLGIRGYEYDYWGQGTLVTVSS

SEQ ID NO: 32 - protein, 130 aa, Artificial Sequence
  antibody sequence
EVQLLESGGGLVQPGGSLRLSCAASGRPFSNYAMGWFRQAPGKEREFVSAISWSGGSTSYADSVKGRFTISRDNAKNTVYLQMNSLRAEDTAVYYCAAQFSGADYGFGRLGIRGYEYDYWGQGTLVTVSS

SEQ ID NO: 33 - protein, 130 aa, Artificial Sequence
  antibody sequence
EVQLLESGGGLVQPGGSLRLSCAASGRPFSNYAMGWFREAPGKEREFVSAISWSGGSTSYADSVKGRFTISRDNSKNTVYLQMNSLRAEDTAVYYCAAQFSGADYGFGRLGIRGYEYDYWGQGTLVTVSS

SEQ ID NO: 34 - protein, 130 aa, Artificial Sequence
  antibody sequence
EVQLLESGGGLVQPGGSLRLSCAASGRPFSNYAMGWFREAPGKEREFVSAISWSGGSTSYADSVKGRFTISRDNAKNTVYLQMNSLRAEDTAVYYCAAQFSGADYGFGRLGIRGYEYDYWGQGTLVTVSS

SEQ ID NO: 35 - protein, 112 aa, Artificial Sequence
  antibody sequence
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSS

SEQ ID NO: 36 - protein, 142 aa, artificial sequence
  antibody sequence
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGLEGHSDHMEQKLISEEDLNRISDHHHHHH

SEQ ID NO: 37 - protein, 277 aa, Artificial Sequence
  antibody sequence
QVQLQESGGGLVQAGGSLRLSCAASGLVFKRYSMNWYRQPPGQQRGLVASISDSGVSTNYADSVKGRFTISRDNAKNIGYLQMNSLKPEDTAVYYCNMHTFWGQGTQVTVSSGGGGSEVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSSGLEGHSDHMEQKLISEEDLNRISDHHHHHH

(19) United States
(12) Patent Application Publication
(10) Pub. No.: US 2022/0135694 A1
(43) Pub. Date: May 5, 2022

(54) NOVEL CD40-BINDING ANTIBODIES

(71) Applicant: LAVA THERAPEUTICS B.V., Utrecht (NL)

(72) Inventors: Johannes Jelle VAN DER VLIET, Utrecht (NL); Iris DE WEERDT, Utrecht (NL); Tanja Denise DE GRUIJL, Utrecht (NL); Paul PARREN, Utrecht (NL); Aron Philip KATER, Utrecht (NL); George Lodewijk SCHEFFER, Utrecht (NL)

(21) Appl. No.: 17/427,291

(22) PCT Filed: Jan. 30, 2020
(86) PCT No.: PCT/NL2020/050051

§ 371 Date: Jul. 30, 2021

(30) Foreign Application Priority Data
Feb. 1, 2019 (NL) 2022494
Oct. 23, 2019 (NL) 2024087

(51) Int. Cl. C07K 16/28; A61P 35/00

(52) U.S. Cl. CPC C07K 16/2878; C07K 16/2809; A61P 35/00; C07K 2317/31; A61K 2039/505; C07K 2317/76; C07K 2317/24; C07K 2317/35; C07K 2317/569

(57) ABSTRACT

The present invention relates to novel antibodies capable of binding human CD40 and to novel multispecific antibodies capable of binding human CD40 and capable of binding a human Vγ9Vδ2 T cell receptor. The invention further relates to pharmaceutical compositions comprising the antibodies of the invention and to uses of the antibodies of the invention for medical treatment.

### FIELD OF THE INVENTION

The present invention relates to novel antibodies capable of binding human CD40 and to novel multispecific antibodies capable of binding human CD40 and capable of binding a human Vγ9Vδ2 T cell receptor. The invention further relates to pharmaceutical compositions comprising the antibodies of the invention and to uses of the antibodies of the invention for medical treatment.

### BACKGROUND OF THE INVENTION

CD40 is a co-stimulatory receptor present on a large number of cell types, including B lymphocytes, dendritic cells, monocytes, endothelial cells, fibroblasts, hematopoietic progenitors, platelets and basal epithelial cells. Binding of the CD40 ligand (CD40L) to CD40 activates intracellular signalling pathways which produce various different biological effects, depending on the cell type and the microenvironment. CD40/CD40L binding plays a role in atherosclerosis, graft rejection, coagulation, infection control and autoimmunity. Many tumor cells also express CD40, including B-cell malignancies and solid tumors, making CD40 a potential target for cancer therapy (Vonderheide (2007) Clin Cancer Res 13:1083).

Both CD40 agonistic as well as CD40 antagonistic drugs have been considered for cancer therapy. CD40 agonists have mostly been chosen, with a 2-fold rationale: First, CD40 agonists can trigger immune stimulation by activating host antigen-presenting cells, which then drive T-cell responses directed against tumors to cause tumor cell death. Second, CD40 ligation can impart direct tumor cytotoxicity on tumors that express CD40 (Vonderheide (2007) Clin Cancer Res 13:1083). Tai et al. (2005) Cancer Res 65: 5898 have described anti-tumor activity of a human antagonistic anti-CD40 antibody (lucatumumab, CHIR-12.12 or HCD 122) against multiple myeloma. A modest activity in relapsed/refractory patients with advanced lymphoma was found (Fanala et al. (2014) Br J Haematol 164:258). A different antagonistic CD40 antibody has been investigated as potential treatment for autoimmune diseases (Schwabe et al. (2018) J Clin Pharmacol, August 16).

While significant progress has been made, no CD40 antibodies have to date been approved for medical use and there is still a need for novel CD40 antibodies that are therapeutically effective yet have acceptable toxicity.

### SUMMARY OF THE INVENTION

The present invention provides novel antibodies for CD40-based therapy. Bispecific antibodies were constructed in which CD40-binding regions were combined with binding regions capable of binding a Vγ9Vδ2 T cell receptor and thus engaging Vγ9Vδ2 T cells. Surprisingly, the bispecific antibodies were able to antagonize CD40 stimulation and efficiently mediate killing of primary chronic lymphocytic leukemia (CLL) cells as well as primary multiple myeloma (MM) cells. Killing was effective even when CLL cells had been stimulated with CD40L. Furthermore, the bispecific antibodies sensitized CLL cells towards venetoclax, a Bcl-2 blocker used in the treatment of CLL.

Bispecific T-cell engaging antibodies having a tumor target binding specificity and a T-cell binding specificity have been described in the art, see e.g. Huehls et al. (2015) Immunol Cell Biol 93:290; Ellerman (2019) Methods, 154:102; de Bruin et al. (2017) Oncoimmunology 7(1):e1375641 and WO2015156673. However, results vary significantly from one tumor target to another. For example, in one study in which a T-cell target (CD3) binding moiety was combined with binding moieties against 8 different B-cell targets (CD20, CD22, CD24, CD37, CD70, CD79b, CD138 and HLA-DR), it was found that the bispecific antibodies targeting the different tumor targets showed strong variation in cytotoxic capacity and cytotoxicity did not correlate with antigen expression levels. For example, CD3-based bispecific antibodies targeting HLA-DR or CD138 were not able to induce cytotoxicity in spite of intermediate to high HLA-DR and CD138 expression levels (Engelberts et al. (2020) Ebiomedicine 52:102625).

In a first aspect, the present invention provides a multispecific antibody comprising a first antigen-binding region capable of binding human CD40 and a second antigen-binding region capable of binding a human Vγ9Vδ2 T cell receptor.

In a second aspect, the invention provides an antibody comprising a first antigen-binding region capable of binding human CD40, wherein the antibody competes for binding to human CD40 with an antibody having the sequence set forth in SEQ ID NO:13 and/or competes for binding to human CD40 with an antibody having the sequence set forth in SEQ ID NO: 14.

In further aspects, the invention relates to pharmaceutical compositions comprising the antibodies of the invention, uses of the antibodies of the invention in medical treatment, and to nucleic acid constructs, expression vectors for producing antibodies of the invention and to host cells comprising such nucleic acid constructs or expression vector.

Further aspects and embodiments of the invention are described below.

### BRIEF DESCRIPTION OF THE DRAWINGS

FIG. 1: Anti-CD40 VHHs bind to CD40-expressing cells. (A) CD40 expression on WT (filled histogram) and CD40-transfected (unfilled histogram) HEK293T cells. (B) CD40-negative WT or CD40-transfected HEK293T cells were incubated with V12t (1 μM), V15t (1 μM), V19t (1 μM) or medium control and the Myc-tag was subsequently detected by flow cytometry. Representative histograms obtained in 3 independent experiments are shown.

FIG. 2: Anti-CD40 VHHs bind to primary CLL cells. (A) CD40 expression on primary CLL cells (black histogram: unstained control, grey histogram: CD40-PE stained). Representative histogram of 5 tested samples is shown. (B) Primary CLL cells (n=5) were incubated with V12t (1 μM), V15t (1 μM), V19t (1 μM) or medium control and the Myc-tag was subsequently detected by flow cytometry. Data represent mean and standard error of mean (SEM). *P<0.05 (B: Repeated-measures one-way ANOVA followed by Dunnett's post hoc test compared to no VHH.)

FIG. 3: The anti-CD40 VHHs are not agonists of CD40. Primary CLL cells (n=6) were cultured with the indicated concentrations of anti-CD40 VHH, rmCD40L (100 ng/mL) or medium control for 48 hours and analyzed by flow cytometry. (A) Viability (B) CD86 and (C) CD95 expression relative to medium control. Data represent mean and SEM. *P<0.05. (A-C: one-way ANOVA followed by Dunnett's post hoc test compared to medium control).

FIG. 4: Monovalent VHHs V15t and V19t antagonize CD40 stimulation. Primary CLL cells (n=6) were pre-incubated with monovalent anti-CD40 VHH or medium control for 30 minutes and then cultured in the presence of recombinant multimeric CD40L (100 ng/mL) for 48 hours and analyzed by flow cytometry. (A) Viability, (B) CD86 and (C) CD95 expression relative to medium control. Data represent mean and SEM. *P<0.05, ***P<0.001, ****P<0.0001. (A-C: one-way ANOVA followed by Dunnett's post hoc test compared to medium control).

FIG. 5: V19S76K-5C8 binds to CD40-expressing cells. CD40-negative WT or CD40-transfected HEK293T cells were incubated with V19S76K-5C8 (1 μM) or medium control and bound bsVHH was detected using anti-llama IgG heavy and light chain antibodies by flow cytometry. Representative histograms obtained in 3 independent experiments are shown.

FIG. 6: V19S76K-5C8 binds to CD40⁺ and Vγ9Vδ2+ cells. Cell lines were incubated with V19S76K-5C8 or medium control and bound bsVHH was detected using anti-llama IgG heavy and light chain antibodies by flow cytometry. (A) Bar plots and (B) non-linear regression analysis of V19S76K-5C8 binding to healthy donor-derived Vγ9Vδ2-T cell lines (n=3). (C) Bar plots and (D) non-linear regression analysis of V19S76K-5C8 binding to healthy donor-derived CD40⁺ CII cell line (n=3). (A, C) data represent mean and SEM; (B, D): data represent mean (symbols), range (error bars), Kd (vertical line) and 95% confidence interval (shaded area). *P<0.05, **P<0.01, ***P<0.001. (A, C: repeated-measures one-way ANOVA followed by Dunnett's post hoc test compared to condition without bsVHH; B, D: non-linear regression analysis).

FIG. 7: V19S76K-5C8 is not an agonist of CD40. Primary CLL cells (n=6) were cultured with the indicated concentrations of V19S76K-5C8, rmCD40L (100 ng/mL) or medium control for 48 hours and analyzed by flow cytometry. (A) CD80, (B) CD86 and (C) CD95 expression relative to medium control. Data represent mean and SEM. *P<0.05. (A-C: repeated-measures one-way ANOVA followed by Dunnett's post hoc test compared to medium control).

FIG. 8: V19S76K-5C8 is an antagonist of CD40. Primary CLL cells (n=6) were pre-incubated with the indicated concentrations of V19S76K-5C8 or medium control for 30 minutes and then cultured in the presence of recombinant multimeric CD40L (100 ng/mL) for 48 hours and analyzed by flow cytometry. (A) CD80, (B) CD86 and (C) CD95 expression relative to medium control. Data represent mean and SEM. *P<0.05, **P<0.01, ***P<0.001, ****P<0.0001. (A-C: repeated-measures one-way ANOVA followed by Dunnett's post hoc test compared to medium control).

FIG. 9: V19S76K-5C8 sensitizes primary CLL cells to venetoclax. Primary CLL cells were pre-incubated with V19S76K-5C8 (1000 nM) or medium control for 30 minutes and then cultured in the presence of recombinant multimeric CD40L (100 ng/mL) for 48 hours. (A) Cells were then cultured with venetoclax (ABT-199) for 24 hours and viability was measured by flow cytometry (n=6). (B) After 48 hours, Bcl-xL expression was analyzed by flow cytometry (n=3). Specific lysis was calculated as: (% cell death in ABT-199 treated cells)−(% cell death in untreated cells)/(% viable cells in untreated cells)*100. Data represent mean and SEM. ***P<0.001, ****P<0.0001. (A: two-way ANOVA followed by Dunnett's post hoc test comparing conditions to medium control, B: repeated-measures one-way ANOVA followed by Dunnett's post hoc test compared to medium control).

FIG. 10: V19S76K-5C8 activates Vγ9Vδ2-T cells. Expanded Vγ9Vδ2-T cells (n=3) were cultured with V19S76K-5C8 and CD40⁺ CII target cells in a 1:1 ratio for 4 hours in the presence of Brefeldin A, monensin and anti-CD107a to measure degranulation and intracellular cytokine production by flow cytometry. (A) CD107a, (B), IFN-γ, (C) TNF-α and (D) IL-2 expression by Vγ9Vδ2-T cells. Data represent mean and SEM. *P<0.05. (A-D: repeated-measures one-way ANOVA followed by Dunnett's post hoc test compared to condition with targets and in the absence of (0 μM) bsVHH).

FIG. 11: V19S76K-5C8 enhances cytotoxicity against CD40⁺ cells. CD40⁺ CII target cells were cultured overnight with expanded Vγ9Vδ2-T cells in a 1:1 ratio in the presence of V19S76K-5C8 and viability was measured by flow cytometry (n=5). (A) Bar plots and (B) non-linear regression analysis of bsVHH-induced cytotoxicity. Cell death is corrected for background cell death in condition without Vγ9Vδ2-T cells by calculating (% cell death in treated cells)−(% cell death in untreated cells)/(% viable cells in untreated cells)*100. (A) Data represent mean and SEM; (B): data represent mean (symbols), range (error bars), Kd (vertical line) and 95% confidence interval (shaded area). *P<0.05, **P<0.01. (A: Repeated-measures one-way ANOVA followed by Dunnett's post hoc test compared to condition with Vγ9Vδ2-T cells and in the absence of (0 nM) bsVHH; B: non-linear regression analysis).

FIG. 12: V19S76K-5C8 cytotoxicity is CD40 specific. Either CD40-negative WT or CD40-transfected HEK293T target cells were cultured overnight with expanded Vγ9Vδ2-T cells in a 1:1 ratio in the presence of V19S76K-5C8. Viability was measured by flow cytometry (n=3). Cell death is corrected for background cell death in the condition without Vγ9Vδ2-T cells by calculating (% cell death in treated cells)−(% cell death in untreated cells)/(% viable cells in untreated cells)*100. Data represent mean and SEM. ****P<0.0001. (mixed effects analysis with Sidak's post hoc test comparing CD40-transfected versus WT mixed effects analysis with Sidak's post hoc test comparing CD40-transfected versus WT).

FIG. 13: V12-5C8t, V15-5C8t and V19-5C8t enhance cytotoxicity against primary CLL cells. CLL target cells were cultured overnight with expanded Vγ9Vδ2-T cells in a 1:1 ratio in the presence of the bispecific VHHs and viability was measured by flow cytometry (n=3). Cell death is corrected for background cell death in condition without Vγ9Vδ2-T cells by calculating (% cell death in treated cells)−(% cell death in untreated cells)/(% viable cells in untreated cells)*100. Data represent mean and SEM. *P<0.05. (two-way ANOVA followed by Tukey's post hoc test comparing mean of each VHH to each other VHH).

FIG. 14: V19S76K-5C8 is effective against CD40-stimulated CLL cells. CLL PBMC samples (n=3) were cultured on irradiated 3T3 or CD40L⁺-3T40L fibroblasts for 72 hours. Cells were then cultured overnight with medium control, healthy donor-derived expanded Vγ9Vδ2-T cells (1:1 ratio), healthy donor-derived expanded Vγ9Vδ2-T cells (1:1 ratio) and V19S76K-5C8 (100 nM), or venetoclax (ABT-199, nM) (n=3). Viability was measured by flow cytometry. Cell death is corrected for background cell death in condition without Vγ9Vδ2-T cells by calculating (% cell death in treated cells)−(% cell death in untreated cells)/(% viable cells in untreated cells)*100. Data represent mean and SEM. ***P<0.001. (Two-way ANOVA followed by Sidak's post hoc test comparing each treatment condition between 3T3 and 3T40L-stimulated CLL cells).

FIG. 15: V19S76K-5C8 activates autologous Vγ9Vδ2-T cells from CLL patients. PBMCs from CLL patients were enriched for T cells by depletion of CD19⁺ CLL cells and then co-cultured with CD19⁺ CLL cells (1:1 ratio) and V19S76K-5C8 (10 nM) or medium control for 16 hours in the presence of Brefeldin A, monensin and anti-CD107a to measure production of (A) IFN-γ, (B) TNF-α, (C) IL-2 and (D) degranulation by flow cytometry (n=7). Data are presented as mean and SEM. *P<0.05, **P<0.01, ***P<0.001. (A-D: paired t-test).

FIG. 16: V19S76K-5C8 induces lysis of autologous CLL cells. CD3⁺ cells and CD19⁺ cells were isolated from PBMC of the same CLL patient and cultured overnight in a 10:1 ratio with V19S76K-5C8 (10 nM) or medium control. Live CLL cells were quantified by flow cytometry using counting beads (n=2 CLL patients). **P<0.01. (Paired t-test).

FIG. 17: V19S76K-5C8 is active against primary multiple myeloma. (A) Example of CD40 expression on primary MM cells, as detected using anti-CD40 PE antibody, clone MAB89, Beckman Couter, IM1936U. Representative histograms of 4 donors (B) Bone marrow of MM patients was cultured overnight in the presence or absence of healthy donor-derived Vγ9Vδ2-T cells in a 1:1 (Vγ9Vδ2-T:plasma cell) ratio in the absence or presence of V19S76K-5C8 (10 μM or 10 nM). Live plasma cells were quantified by flow cytometry using counting beads (n=5). (C, D) Mononuclear cells from the bone marrow of MM patients were cultured overnight with V19S76K-5C8 (VHH; 10 nM), aminobisphosphonate (ABP; 10 μM zoledronic acid (positive control)) or medium control in the presence of brefeldin, monensin and anti-CD107a to measure (C) cytokine production and (D) degranulation by flow cytometry (n=6). Data are presented as mean and SEM. *P<0.05, **P<0.01. (B-D: repeated-measures one-way ANOVA followed by Dunnett's post hoc test compared to condition without antibody).

FIG. 18: The bispecific anti-CD40-V62 VHH prolongs survival in vivo. Immunodeficient NSG mice were irradiated on day −1 and grafted (i.v.) with 2.5*10⁶ MM.1s cells on day 0. Mice received PBS or human Vγ9Vδ2-T cells (1*10⁷ cells; both i.v.) on days 7, 14 and 21 followed by PBS or V19S76K-5C8 (VHH; 5 mg/kg; both i.p.) twice weekly starting on day 9. (A) Schematic overview of treatment schedule. (B) Kaplan-Meier analyses of mouse survival (control: n=6; V19S76K-5C8 (VHH): n=6, Vγ9Vδ2-T cells: n=8, Vγ9Vδ2-T cells+V19S76K-5C8 (VHH): n=8). CD40 expression on MM.1s cells (human CD45⁺CD38⁺ cells) in the (C) bone marrow (BM) and (D) plasmacytomas at the time of sacrifice. (E) Body weight after 7 weeks of treatment relative to individual body weight at time of tumor injection. **P<0.01, ***P<0.001. Data are presented as mean and SD. (B: Mantel-Cox logrank test followed by Holm-Sidak's post hoc test, C: one-way ANOVA followed by Dunnett's post hoc test compared to control mice, D: unpaired t-test).

### DETAILED DESCRIPTION OF THE INVENTION

### Definitions

The term “human CD40”, when used herein, refers to the CD40 protein, also known as tumor necrosis factor receptor superfamily member 5 (UniProtKB—P25942 (TNR5_HUMAN)), Isoform I, set forth in SEQ ID NO:24.

The term “human Vδ2”, when used herein, refers to the TRDV2 protein, T cell receptor delta variable 2 (UniProtKB—AOJD36 (AOJD36_HUMAN) gives an example of a Vδ2 sequence).

The term “human Vγ9”, when used herein, refers to the TRGV9 protein, T cell receptor gamma variable 9 (UniProtKB—Q99603_HUMAN gives an example of a Vγ9 sequence).

The term “antibody” is intended to refer to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The antigen-binding region (or antigen-binding domain) which interacts with an antigen may comprise variable regions of both the heavy and light chains of the immunoglobulin molecule or may be a single-domain antigen-binding region, e.g. a heavy chain variable region only.

The constant regions of an antibody, if present, may mediate the binding of the immunoglobulin to host tissues or factors, including various cells of the immune system (such as effector cells and T cells) and components of the complement system such as C1q, the first component in the classical pathway of complement activation. In some embodiments, however, the Fc region of the antibody has been modified to become inert, “inert” means an Fc region which is at least not able to bind any Fcγ Receptors, induce Fc-mediated cross-linking of FcRs, or induce FcR-mediated cross-linking of target antigens via two Fc regions of individual antibodies. In a further embodiment, the inert Fc region is in addition not able to bind C1q. In one embodiment, the antibody contains mutations at positions 234 and 235 (Canfield and Morrison (1991) J Exp Med 173:1483), e.g. a Leu to Phe mutation at position 234 and a Leu to Glu mutation at position 235. In another embodiment, the antibody contains a Leu to Ala mutation at position 234, a Leu to Ala mutation at position 235 and a Pro to Gly mutation at position 329. In another embodiment, the antibody contains a Leu to Phe mutation at position 234, a Leu to Glu mutation at position 235 and an Asp to Ala at position 265.

As indicated above, the term antibody as used herein, unless otherwise stated or clearly contradicted by context, includes fragments of an antibody that retain the ability to specifically bind to the antigen. It has been shown that the antigen-binding function of an antibody may be performed by fragments of a full-length antibody. Examples of binding fragments encompassed within the term “antibody” include (i) a Fab′ or Fab fragment, i.e. a monovalent fragment consisting of the VL, VH, CL and CH1 domains, or a monovalent antibody as described in WO2007059782; (ii) F(ab′)2 fragments, i.e. bivalent fragments comprising two Fab fragments linked by a disulfide bridge at the hinge region; (iii) a Fd fragment consisting essentially of the VH and CH1 domains; and (iv) a Fv fragment consisting essentially of the VL and VH domains of a single arm of an antibody. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise indicated by context. Although such fragments are generally included within the meaning of antibody, they collectively and each independently are unique features of the present invention, exhibiting different biological properties and utility. The term antibody, unless specified otherwise, also includes polyclonal antibodies, monoclonal antibodies (mAbs), chimeric antibodies and humanized antibodies, and antibody fragments provided by any known technique, such as enzymatic cleavage, peptide synthesis, and recombinant techniques.

In some embodiments of the antibodies of the invention, the first antigen-binding region or the antigen-binding region, or both, is a single domain antibody. Single domain antibodies (sdAb, also called Nanobody®, or VHH) are well known to the skilled person, see e.g. Hamers-Casterman et al. (1993) Nature 363:446, Roovers et al. (2007) Curr Opin Mol Ther 9:327 and Krah et al. (2016) Immunopharmacol Immunotoxicol 38:21. Single domain antibodies comprise a single CDR1, a single CDR2 and a single CDR3. Examples of single domain antibodies are variable fragments of heavy-chain-only antibodies, antibodies that naturally do not comprise light chains, single domain antibodies derived from conventional antibodies, and engineered antibodies. Single domain antibodies may be derived from any species including mouse, human, camel, llama, shark, goat, rabbit, and cow. For example, naturally occurring VHH molecules can be derived from antibodies raised in Camelidae species, for example in camel, dromedary, alpaca and guanaco. Like a whole antibody, a single domain antibody is able to bind selectively to a specific antigen. Single domain antibodies may contain only the variable domain of an immunoglobulin chain, i.e. CDR1, CDR2 and CDR3 and framework regions.

The term “immunoglobulin” as used herein is intended to refer to a class of structurally related glycoproteins consisting of two pairs of polypeptide chains, one pair of light (L) chains and one pair of heavy (H) chains, all four potentially inter-connected by disulfide bonds. The term “immunoglobulin heavy chain”, “heavy chain of an immunoglobulin” or “heavy chain” as used herein is intended to refer to one of the chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The heavy chain constant region further comprises a hinge region. Within the structure of the immunoglobulin (e.g. IgG), the two heavy chains are inter-connected via disulfide bonds in the hinge region. Equally to the heavy chains, each light chain is typically comprised of several regions; a light chain variable region (VL) and a light chain constant region (CL). Furthermore, the VH and VL regions may be subdivided into regions of hypervariability (or hypervariable regions which may be hypervariable in sequence and/or form structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences may be determined by use of various methods, e.g. the methods provided by Choitia and Lesk (1987) J. Mol. Biol. 196:901 or Kabat et al. (1991) Sequence of protein of immunological interest, fifth edition. NIH publication. Various methods for CDR determination and amino acid numbering can be compared on www.abysis.org (UCL).

The term “isotype” as used herein, refers to the immunoglobulin (sub)class (for instance IgG1, IgG2, IgG3, IgG4, IgD, IgA, IgE, or IgM) or any allotype thereof, such as IgG1m(za) and IgG1m(f) that is encoded by heavy chain constant region genes. Each heavy chain isotype can be combined with either a kappa (κ) or lambda (Δ) light chain. An antibody of the invention can possess any isotype.

The term “full-length antibody” when used herein, refers to an antibody which contains all heavy and light chain constant and variable domains corresponding to those that are normally found in a wild-type antibody of that isotype.

The term “chimeric antibody” refers to an antibody wherein the variable region is derived from a non-human species (e.g. derived from rodents) and the constant region is derived from a different species, such as human. Chimeric antibodies may be generated by genetic engineering. Chimeric monoclonal antibodies for therapeutic applications are developed to reduce antibody immunogenicity.

The term “humanized antibody” refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and, optionally, fully human constant regions. Optionally, additional amino acid modifications, which are not necessarily back-mutations, may be introduced to obtain a humanized antibody with preferred characteristics, such as affinity and biochemical properties. Humanization of non-human therapeutic antibodies is performed to minimize its immunogenicity in man while such humanized antibodies at the same time maintain the specificity and binding affinity of the antibody of non-human origin.

The term “multispecific antibody” refers to an antibody having specificities for at least two different, such as at least three, typically non-overlapping, epitopes. Such epitopes may be on the same or on different target antigens. If the epitopes are on different targets, such targets may be on the same cell or different cells or cell types.

The term “bispecific antibody” refers to an antibody having specificities for two different, typically non-overlapping, epitopes. Such epitopes may be on the same or different targets. If the epitopes are on different targets, such targets may be on the same cell or different cells or cell types.

Examples of different classes of bispecific antibodies include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) ScFv- and diabody-based and heavy chain antibodies (e.g., domain antibodies, Nanobodies®) wherein different single chain Fv molecules or different diabodies or different heavy-chain antibodies (e.g. domain antibodies, Nanobodies®) are fused to each other or to another protein or carrier molecule fused to heavy-chain constant-domains, Fc-regions or parts thereof.

Examples of IgG-like molecules with complementary CH3 domains molecules include but are not limited to the Triomab® (Trion Pharma/Fresenius Biotech), the Knobs-into-Holes (Genentech), CrossMAbs (Roche) and the electrostatically-matched (Amgen, Chugai, Oncomed), the LUZ-Y (Genentech, Wranik et al. J. Biol. Chem. 2012, 287(52): 43331-9, doi: 10.1074/jbc.M112.397869. Epub 2012 November 1), DIG-body and PIG-body (Pharmabcine, WO2010134666, WO2014081202), the Strand Exchange Engineered Domain body (SEEDbody)(EMD Serono), the Biclonics (Merus, WO2013157953), FcAAdp (Regeneron), bispecific IgG1 and IgG2 (Pfizer/Rinat), Azymetric scaffold (Zymeworks/Merck,), mAb-Fv (Xencor), bivalent bispecific antibodies (Roche, WO2009080254) and DuoBody® molecules (Genmab).

Examples of recombinant IgG-like dual targeting molecules include but are not limited to Dual Targeting (DT)-Ig (GSK/Domantis, WO2009058383), Two-in-one Antibody (Genentech, Bostrom, et al 2009. Science 323, 1610-1614), Cross-linked Mabs (Karmanos Cancer Center), mAb2 (F-Star), Zybodies™ (Zyngenia, LaFleur et al. MAbs. 2013 March-April; 5(2):208-18), approaches with common light chain, KABodies (NovImmune, WO2012023053) and CovX-body® (CovX/Pfizer, Doppalapudi, V. R., et al 2007. Bioorg. Med. Chem. Lett. 17, 501-506).

Examples of IgG fusion molecules include but are not limited to Dual Variable Domain (DVD)-Ig (Abbott), Dual domain double head antibodies (Unilever; Sanofi Aventis), IgG-like Bispecific (ImClone/Eli Lilly, Lewis et al. Nat Biotechnol. 2014 February; 32(2):191-8), Ts2Ab (MedImmune/AZ, Dimasi et al. J Mol Biol. 2009 Oct. 30; 393(3):672-92) and BsAb (Zymogenetics, WO2010111625), HERCULES (Biogen Idec), scFv fusion (Novartis), scFv fusion (Changzhou Adam Biotech Inc) and TvAb (Roche).

Examples of Fc fusion molecules include but are not limited to ScFv/Fc Fusions (Academic Institution, Pearce et al Biochem Mol Biol Int. 1997 September; 42(6):1179), SCORPION (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART™) (MacroGenics) and Dual(ScFv)2-Fab (National Research Center for Antibody Medicine—China).

Examples of Fab fusion bispecific antibodies include but are not limited to F(ab)2 (Medarex/AMGEN), Dual-Action or Bis-Fab (Genentech), Dock-and-Lock® (DNL) (ImmunoMedics), Bivalent Bispecific (Biotecnol) and Fab-Fv (UCB-Celltech).

Examples of ScFv-, diabody-based and domain antibodies include but are not limited to Bispecific T Cell Engager (BiTE®) (Micromet, Tandem Diabody (Tandab) (Affimed), Dual Affinity Retargeting Technology (DART™) (MacroGenics), Single-chain Diabody (Academic, Lawrence FEBS Lett. 1998 Apr. 3; 425(3):479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 August; 88(6):667-75), dual targeting Nanobodies® (Ablynx, Hmila et al., FASEB J. 2010), dual targeting heavy chain only domain antibodies.

In the context of antibody binding to an antigen, the terms “binds” or “specifically binds” refer to the binding of an antibody to a predetermined antigen or target (e.g. human CD40 or Vδ2) to which binding typically is with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less, e.g. when determined using flow cytometry as described in the Examples herein. Alternatively, apparent K_{D} values can be determined using by for instance surface plasmon resonance (SPR) technology in a BIAcore 3000 instrument using the antigen as the ligand and the binding moiety or binding molecule as the analyte. Specific binding means that the antibody binds to the predetermined antigen with an affinity corresponding to a K_{D} that is at least ten-fold lower, such as at least 100-fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The degree with which the affinity is lower is dependent on the K_{D} of the binding moiety or binding molecule, so that when the K_{D} of the binding moiety or binding molecule is very low (that is, the binding moiety or binding molecule is highly specific), then the degree with which the affinity for the antigen is lower than the affinity for a non-specific antigen may be at least 10,000-fold. The term “K_{D}” (M), as used herein, refers to the dissociation equilibrium constant of a particular interaction between the antigen and the binding moiety or binding molecule.

In the context of the present invention, “competition” or “able to compete” or “competes” refers to any detectably significant reduction in the propensity for a particular binding molecule (e.g. a CD40 antibody) to bind a particular binding partner (e.g. CD40) in the presence of another molecule (e.g. a different CD40 antibody) that binds the binding partner. Typically, competition means an at least about 25 percent reduction, such as an at least about 50 percent, e.g. an at least about 75 percent, such as an at least 90 percent reduction in binding, caused by the presence of another molecule, such as an antibody, as determined by, e.g., ELISA analysis or flow cytometry using sufficient amounts of the two or more competing molecules, e.g. antibodies. Additional methods for determining binding specificity by competitive inhibition may be found in for instance Harlow et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc, and Wiley InterScience N. Y., (1992, 1993), and Muller, Meth. Enzymol. 92, 589-601 (1983)). In one embodiment, the antibody of the present invention binds to the same epitope on CD40 as antibody V15 or V19 and/or to the same epitope on Vδ2 as antibody 5C8 or 6H4. Methods for determining the epitope of a binding molecule, such as an antibody, are known in the art.

The terms “first” and “second” antigen-binding regions when used herein do not refer to their orientation/position in the antibody, i.e. it has no meaning with regard to the N- or C-terminus. The term “first” and “second” only serves to correctly and consistently refer to the two different antigen-binding regions in the claims and the description.

“Capable of binding a Vγ9Vδ2-TCR” means that the binding molecule can bind a Vγ9Vδ2-TCR, but does not exclude that the binding molecule binds to one of the separate subunits in the absence of the other subunit, i.e. to the Vγ9 chain alone or to the Vδ2 chain alone. For example, antibody 5C8 is an antibody that binds the Vγ9Vδ2-TCR, but also binds the Vδ2 chain when the Vδ2 chain is expressed alone.

“% sequence identity”, when used herein, refers to the number of identical nucleotide or amino acid positions shared by different sequences (i.e., % identity=# of identical positions/total # of positions×100), taking into account the number of gaps, and the length of each gap, which need to be introduced for optimal alignment. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4.

### Further Aspects and Embodiments of the Invention

As described above, in a first main aspect, the invention relates to a multispecific antibody comprising a first antigen-binding region capable of binding human CD40 and a second antigen-binding region capable of binding a human Vγ9Vδ2-T cell receptor.

In one embodiment, the multispecific antibody is a bispecific antibody. In another embodiment, the first antigen-binding region is a single-domain antibody. In another embodiment, the second antigen-binding region is a single-domain antibody. In a further embodiment, both the first antigen-antigen binding region and the second antigen-binding region are single-domain antibodies.

In one embodiment, the first antigen-binding region and the second antigen-binding region are covalently linked to each other via a peptide linker, e.g. a linker having a length of from 1 to 20 amino acids, e.g. from 1 to 10 amino acids, such as 2, 3, 4, 5, 6, 7, 8 or 10 amino acids. In one embodiment, the peptide linker comprises or consists of the sequence GGGGS, set forth in SEQ ID NO: 21.

In one embodiment of the multispecific antibody, the first antigen-binding region is located N-terminally of the second antigen-binding region.

In one embodiment, the multispecific antibody binds monovalently to CD40 and binds monovalently to the human Vγ9Vδ2 T cell receptor.

In one embodiment of the multispecific antibody of the invention, the multispecific antibody is not an agonist of human CD40. CD40 agonism may be tested by determining the ability of the antibody to increasing the level of expression of CD80, CD86 and/or CD95 on CD40-expressing cells, e.g. primary cells from a CLL patient. Such an assay may be performed as described in Example 8 herein. In one embodiment, the expression of CD80 on primary cells from a CLL patient is less than 10%, such as less than 5%, increased in the presence of antibody as compared to a control wherein the antibody is absent. In another embodiment, the expression of CD86 on primary cells from a CLL patient is less than 10%, such as less than 5%, increased in the presence of antibody as compared to a control wherein the antibody is absent. In a further embodiment, the expression of CD95 on primary cells from a CLL patient is less than 10%, such as less than 5%, increased in the presence of antibody as compared to a control wherein the antibody is absent.

In a further embodiment of the multispecific antibody of the invention, the multispecific antibody is an antagonist of human CD40. An antagonistic effect on CD40 may e.g. be determined by testing the ability of an antibody to inhibit the activation of CD40 by CD40L on CD40-expressing cells, e.g. primary cells from a CLL patient. Such an assay may be performed as described in Example 9 herein. In one embodiment, the expression of CD80 on primary cells from a CLL patient in the presence of sufficient concentrations of CD40L is less than 20%, such as less than 10%, increased in the presence of antibody as compared to a control wherein the antibody is absent. In one embodiment, the expression of CD86 on primary cells from a CLL patient in the presence of sufficient concentrations of CD40L is less than 20%, such as less than 10%, increased in the presence of antibody as compared to a control wherein the antibody is absent. In one embodiment, the expression of CD95 on primary cells from a CLL patient in the presence of sufficient concentrations of CD40L is less than 20%, such as less than 10%, increased in the presence of antibody as compared to a control wherein the antibody is absent.

In a further embodiment, the multispecific antibody is capable of sensitizing human CD40-expressing cells, e.g. primary cells from a CLL patient, to venetoclax. Sensitization of primary cells from a CLL patient towards venetoclax by an antibody may be assessed by determining primary cell viability in the presence of various concentrations of venetoclax in the presence or absence of antibody. Such an assay may be performed as described in Example 10 herein. In one embodiment, the specific cell death at a venetoclax concentration of 100 nM is at least 10%, such as at least 20% higher in the presence of the antibody as compared to a control where the antibody is absent, when assayed as described in Example 10 herein.

In a further embodiment, the multispecific antibody binds CD40⁺ CII cells with a Kd below 200 nM, e.g. below 100 nM, such as below 50 nM, e.g. below 20 nM, such as between 5 and 15 nM, e.g. when tested as described in Example 7 herein.

In a further embodiment, the multispecific antibody competes (i.e. is able to compete) for binding to human CD40 with an antibody having the sequence set forth in SEQ ID NO:13 and/or competes for binding to human CD40 with an antibody having the sequence set forth in SEQ ID NO:14.

In a further embodiment, the multispecific antibody binds the same epitope on human CD40 as an antibody having the sequence set forth in SEQ ID NO: 13 or binds the same epitope on human CD40 as antibody having the sequence set forth in SEQ ID NO:14.

In a further embodiment, the first antigen-binding region comprises:
the VH CDR1 sequence set forth in SEQ ID NO:1, the VH CDR2 sequence set forth in SEQ ID NO:2 and the VH CDR3 sequence set forth in SEQ ID NO:3, or
the VH CDR1 sequence set forth in SEQ ID NO:4, the VH CDR2 sequence set forth in SEQ ID NO:5 and the VH CDR3 sequence set forth in SEQ ID NO:6.

In one embodiment, the first antigen-binding region is humanized. In another embodiment, the first antigen-binding region comprises or consists of:
the sequence set forth in SEQ ID NO:13 or the sequence set forth in SEQ ID NO:14, or
a sequence having at least 90%, such as least 92%, e.g. at least 94%, such as at least 96%, e.g. at least 98% sequence identity to the sequence set forth in SEQ ID NO: 13 or a sequence having at least 90%, such as least 92%, e.g. at least 94%, such as at least 96%, e.g. at least 98% sequence identity to the sequence set forth in SEQ ID NO: 14.

As described above, the multispecific antibody of the invention comprises a second antigen-binding region capable of binding a human Vγ9Vδ2-T cell receptor. In one embodiment, the multispecific antibody is able to activate human Vγ9Vδ2 T cells. The activation of the Vγ9Vδ2 T cells may be measured through gene-expression and/or (surface) marker expression (e.g., activation markers, such as CD25, CD69, or CD107a) and/or secretory protein (e.g., cytokines or chemokines) profiles. In a preferred embodiment, the multispecific antibody is able to induce activation (e.g. upregulation of CD69 and/or CD25 expression) resulting in degranulation marked by an increase in CD107a expression, see Example 11) and cytokine production (e.g. TNFα, IFNγ) by Vγ9Vδ2 T cells. Preferably, a multispecific antibody of the present invention is able to increase the number of cells positive for CD107a at least 1.5-fold, such as at least 2-fold, e.g. at least 5-fold.

In a further embodiment, the multispecific antibody is capable of mediating killing of human CD40-expressing cells from a chronic lymphocytic leukemia patient. Killing of human CD40-expressing cells from a chronic lymphocytic leukemia patient may e.g. be determined as described in Example 12 herein. In one embodiment, the multispecific antibody of the invention is capable of mediating specific cell death of more than 25%, such as more than 30%, at a concentration of 10 pM, as determined in the assay described in Example 12 herein. In a further embodiment, the multispecific antibody when assayed as described in Example 12 herein has a half maximal effective concentration between 1 and 20 pM, e.g. between 5 and 10 pM.

In a further embodiment, the multispecific antibody is capable of mediating killing of CD40-expressing cells from a chronic lymphocytic leukemia patient that have been stimulated with CD40L. Killing of CD40L-stimulated CD40-expressing cells from a chronic lymphocytic leukemia patient may e.g. be determined as described in Example 15 herein. In one embodiment, the multispecific antibody of the invention is capable of mediating specific cell death of more than 25%, such as more than 50%, at a concentration of 10 nM, as determined in the assay described in Example 15 herein.

In a further embodiment, the multispecific antibody is capable of mediating lysis of human CD40-expressing cells from a multiple myeloma patient. Lysis of human CD40-expressing cells from a multiple myeloma patient may e.g. be determined as described in Example 18 herein. In one embodiment, the multispecific antibody of the invention is capable of mediating specific cell lysis of more than 25%, such as more than 40%, at a concentration of 10 nM, as determined in the assay described in Example 18 herein.

In one embodiment of the multispecific antibody of the invention, the multispecific antibody is capable of binding to human Vδ2. Vδ2 is the delta chain of the Vγ9Vδ2-TCR. In another embodiment, the multispecific antibody is capable of binding to human Vγ9. Vγ9 is the gamma chain of Vγ9Vδ2-TCR. Several such antibodies which bind to Vδ2 or Vγ9 have been described in WO2015156673 and their antigen-binding regions at least the CDR sequences thereof can be incorporated in the multispecific antibody of the invention. Other examples of antibodies from which a Vγ9Vδ2-TCR-binding region might be derived are TCR Vγ9 antibody 7A5 (ThermoFisher) (Oberg et al. (2014) Cancer Res 74:1349) and antibodies B1.1 (ThermoFisher) and 5A6.E9 (ATCC HB 9772), both described in Neuman et al. (2016) J Med Prim 45:139.

In one embodiment, the multispecific antibody binds to Vγ9Vδ2⁺ T cells with a Kd below 100 nM, e.g. below 50 nM, such as below 20 nM, e.g. below 10 nM, such as between 0.5 and 2.5 nM, e.g. when tested as described in Example 7 herein.

In one embodiment, the multispecific antibody competes for binding to human Vδ2 with an antibody having the sequence set forth in SEQ ID NO: 17 or competes for binding to human Vδ2 with an antibody having the sequence set forth in SEQ ID NO: 18. In a further embodiment, the multispecific antibody binds the same epitope on human Vδ2 as an antibody having the sequence set forth in SEQ ID NO: 17 or binds the same epitope on human Vδ2 as an antibody having the sequence set forth in SEQ ID NO: 18.

In one embodiment of the multispecific antibody of the invention, the second antigen-binding region comprises the VH CDR1 sequence set forth in SEQ ID NO:7, the VH CDR2 sequence set forth in SEQ ID NO:8 and the VH CDR3 sequence set forth in SEQ ID NO:9 or comprises the VH CDR1 sequence set forth in SEQ ID NO:10, the VH CDR2 sequence set forth in SEQ ID NO:11 and the VH CDR3 sequence set forth in SEQ ID NO: 12.

In another embodiment of the multispecific antibody of the invention, the second antigen-binding region comprises the VH CDR1 sequence set forth in SEQ ID NO:10, the VH CDR2 sequence set forth in SEQ ID NO:11 and the VH CDR3 sequence set forth in SEQ ID NO: 12.

In one embodiment of the multispecific antibody of the invention, the second antigen-binding region is humanized.

In a further embodiment, the second antigen-binding region comprises or consists of
the sequence set forth in SEQ ID NO:17, or
a sequence having at least 90%, such as least 92%, e.g. at least 94%, such as at least 96%, e.g. at least 98% sequence identity to the sequence set forth in SEQ ID NO: 17, or
a sequence selected from the group consisting of SEQ ID NO: 25, 26, 27, 28, 29, 30, 31, 32, 33 and 34.

In one embodiment of the multispecific antibody of the invention, the first antigen-binding region comprises
the VH CDR1 sequence set forth in SEQ ID NO:1, the VH CDR2 sequence set forth in SEQ ID NO:2 and the VH CDR3 sequence set forth in SEQ ID NO:3, or
the VH CDR1 sequence set forth in SEQ ID NO:4, the VH CDR2 sequence set forth in SEQ ID NO:5 and the VH CDR3 sequence set forth in SEQ ID NO: 6, and the second antigen-binding region comprises the VH CDR1 sequence set forth in SEQ ID NO:7, the VH CDR2 sequence set forth in SEQ ID NO:8 and the VH CDR3 sequence set forth in SEQ ID NO:9.

In another embodiment of the multispecific antibody of the invention, the first antigen-binding region comprises
the VH CDR1 sequence set forth in SEQ ID NO:1, the VH CDR2 sequence set forth in SEQ ID NO:2 and the VH CDR3 sequence set forth in SEQ ID NO:3, or
the VH CDR1 sequence set forth in SEQ ID NO:4, the VH CDR2 sequence set forth in SEQ ID NO:5 and the VH CDR3 sequence set forth in SEQ ID NO:6, and the second antigen-binding region comprises the VH CDR1 sequence set forth in SEQ ID NO:10, the VH CDR2 sequence set forth in SEQ ID NO:11 and the VH CDR3 sequence set forth in SEQ ID NO: 12.

As described above, in a further main aspect, the invention relates to an antibody comprising a first antigen-binding region capable of binding human CD40, wherein the antibody competes for binding to human CD40 with an antibody having the sequence set forth in SEQ ID NO:13 and/or competes for binding to human CD40 with an antibody having the sequence set forth in SEQ ID NO: 14.

In one embodiment, the antibody binds the same epitope on human CD40 as an antibody having the sequence set forth in SEQ ID NO:13 or binds the same epitope on human CD40 as antibody having the sequence set forth in SEQ ID NO: 14.

In a further embodiment, the first antigen-binding region comprises:
the VH CDR1 sequence set forth in SEQ ID NO:1, the VH CDR2 sequence set forth in SEQ ID NO:2 and the VH CDR3 sequence set forth in SEQ ID NO:3, or
the VH CDR1 sequence set forth in SEQ ID NO:4, the VH CDR2 sequence set forth in SEQ ID NO:5 and the VH CDR3 sequence set forth in SEQ ID NO:6.

In an even further embodiment, the first antigen-binding region comprises or consists of:
the sequence set forth in SEQ ID NO:13 or the sequence set forth in SEQ ID NO:14, or
a sequence having at least 90%, such as least 92%, e.g. at least 94%, such as at least 96%, e.g. at least 98% sequence identity to the sequence set forth in SEQ ID NO:13 or a sequence having at least 90%, such as least 92%, e.g. at least 94%, such as at least 96%, e.g. at least 98% sequence identity to the sequence set forth in SEQ ID NO: 14.

In a further embodiment, the first antigen-binding region is a single-domain antibody. In another embodiment, the antibody is a monospecific antibody, e.g. a monovalent antibody. In a further embodiment, the antibody comprises a second antigen-binding region which binds an antigen which is not human CD40 or Vδ2.

In a further embodiment, the antibody is not an agonist of human CD40. As mentioned, CD40 agonism may be tested by determining the ability of the antibody to increasing the level of expression of CD80, CD86 and/or CD95 on CD40-expressing cells, e.g. primary cells from a CLL patient. Such an assay may be performed as described in Example 4 herein. In one embodiment, the expression of CD80 on primary cells from a CLL patient is less than 10%, such as less than 5%, increased in the presence of antibody as compared to a control wherein the antibody is absent. In another embodiment, the expression of CD86 on primary cells from a CLL patient is less than 10%, such as less than 5%, increased in the presence of antibody as compared to a control wherein the antibody is absent. In a further embodiment, the expression of CD95 on primary cells from a CLL patient is less than 10%, such as less than 5%, increased in the presence of antibody as compared to a control wherein the antibody is absent.

In a further embodiment, the antibody is an antagonist of human CD40. As mentioned, an antagonistic effect on CD40 may e.g. be determined by testing the ability of an antibody to inhibit the activation of CD40 by CD40L on CD40-expressing cells, e.g. primary cells from a CLL patient. Such an assay may be performed as described in Example 5 herein. In one embodiment, the expression of CD80 on primary cells from a CLL patient in the presence of sufficient concentrations of CD40L is less than 20%, such as less than 10%, increased in the presence of antibody as compared to a control wherein the antibody is absent. In one embodiment, the expression of CD86 on primary cells from a CLL patient in the presence of sufficient concentrations of CD40L is less than 20%, such as less than 10%, increased in the presence of antibody as compared to a control wherein the antibody is absent. In one embodiment, the expression of CD95 on primary cells from a CLL patient in the presence of sufficient concentrations of CD40L is less than 20e, such as less than 10, increased in the presence of antibody as compared to a control wherein the antibody is absent.

In a further embodiment, the antibody is capable of sensitizing human CD40-expressing cells, e.g. primary cells from a CLL patient, to venetoclax. Sensitization of primary cells from a CLL patient towards venetoclax by an antibody may be assessed by determining primary cell viability in the presence of various concentrations of venetoclax in the presence or absence of antibody. Such an assay may be performed as described in Example 10 herein. In one embodiment, the specific cell death at a venetoclax concentration of 100 nM is at least 10%, such as at least 200/higher in the presence of the antibody as compared to a control where the antibody is absent, when assayed as described in Example 10 herein.

TABLE 1
Sequence listing.

| SEQ | | Descrip- | |
|---|---|---|---|
| ID. | code | tion | Sequence |
| 1 | V19 | CDR1 | RSAMG |
| 2 | V19 | CDR2 | AIGTRGGSTKYADSVKG |
| 3 | V19 | CDR3 | RGPGYPSAAIFQDEYHY |
| 4 | V15 | CDR1 | SDTMG |
| 5 | V15 | CDR2 | SISSRGVREYADSVKG |
| 6 | V15 | CDR3 | GALGLPGYRPYNN |
| 7 | 5C8 | CDR1 | NYAMG |
| 8 | 5C8 | CDR2 | AISWSGGSTSYADSVKG |
| 9 | 5C8 | CDR3 | QFSGADYGFGRLGIRGYEYDY |
| 10 | 6H4 | CDR1 | NYGMG |
| 11 | 6H4 | CDR2 | GISWSGGSTDYADSVKG |
| 12 | 6H4 | CDR3 | VFSGAETAYYPSDDYDY |
| 13 | V19 | VHH | QVQLQESGGGLVQAGGSLRLS |
| | | | CAASGRTFGRSAMGWFRQAPG |
| | | | KEREFVAAIGTRGGSTKYADS |
| | | | VKGRFTISTDNASNTVYLQMD |
| | | | SLKPEDTAVYRCAVRGPGYPS |
| | | | AAIFQDEYHYWGQGTQVTVSS |
| 14 | V15 | VHH | EVQLQESGGGLVQAGGSLRLS |
| | | | CVTSGSAFSSDTMGWFRQAPG |
| | | | KQRELVASISSRGVREYADSV |
| | | | KGRFTISRDNAKNTVYLQMNS |
| | | | LQPEDTAVYYCNRGALGLPGY |
| | | | RPYNNWGQGTQVTVSS |
| 15 | V19t | VHH | QVQLQESGGGLVQAGGSLRLS |
| | | | CAASGRTFGRSAMGWFRQAPG |
| | | | KEREFVAAIGTRGGSTKYADS |
| | | | VKGRFTISTDNASNTVYLQMD |
| | | | SLKPEDTAVYRCAVRGPGYPS |
| | | | AAIFQDEYHYWGQGTQVTVSS |
| | | | GLEGHSDHMEQKLISEEDLNR |
| | | | ISDHHHHHH |
| 16 | V15t | VHH | EVQLQESGGGLVQAGGSLRLS |
| | | | CVTSGSAFSSDTMGWFRQAPG |
| | | | KQRELVASISSRGVREYADSV |
| | | | KGRFTISRDNAKNTVYLQMNS |
| | | | LQPEDTAVYYCNRGALGLPGY |
| | | | RPYNNWGQGTQVTVSSGLEGH |
| | | | SDHMEQKLISEEDLNRISDHH |
| | | | HHHH |
| 17 | 5C8 | VHH | EVQLVESGGGLVQAGGSLRLS |
| | | | CAASGRPFSNYAMGWFRQAPG |
| | | | KEREFVAAISWSGGSTSYADS |
| | | | VKGRFTISRDNAKNTVYLQMN |
| | | | SPKPEDTAIYYCAAQFSGADY |
| | | | GFGRLGIRGYEYDYWGQGTQV |
| | | | TVSS |
| 18 | 6H4 | VHH | EVQLVESGGGLVQAGGSLRLS |
| | | | CAASGRPFSNYGMGWFRQAPG |
| | | | KKREFVAGISWSGGSTDYADS |
| | | | VKGRFTISRDNAKNTVYLQMN |
| | | | SLKPEDTAVYYCAAVFSGAET |
| | | | AYYPSDDYDYWGQGTQVTVSS |
| 19 | V19- | Bispecific | QVQLQESGGGLVQAGGSLRLS |
| | 5C8t | binding | CAASGRTFGRSAMGWFRQAPG |
| | | molecule | KEREFVAAIGTRGGSTKYADS |
| | | | VKGRFTISTDNASNTVYLQMD |
| | | | SLKPEDTAVYRCAVRG |
| | | | PGYPSAAIFQDEYHYWGQGTQ |
| | | | VTVSSGGGGSEVQLVESGGGL |
| | | | VQAGGSLRLSCAASGRPFSNY |
| | | | AMGWFRQAPGKEREFVAAISW |
| | | | SGGSTSYADSVKGRFTISRDN |
| | | | AKNTVYLQMNSPKPEDTAIYY |
| | | | CAAQFSGADYGFGRLGIRGYE |
| | | | YDYWGQGTQVTVSSGLEGHSD |
| | | | HMEQKLISEEDLNRISDHHHH |
| | | | HH |
| 20 | V15- | Bispecific | EVQLQESGGGLVQAGGSLRLS |
| | 5C8t | binding | CVTSGSAFSSDTMGWFRQAPG |
| | | molecule | KQRELVASISSRGVREYADSV |
| | | | KGRFTISRDNAKNTVYLQMNS |
| | | | LQPEDTAVYYCNRGALGLPGY |
| | | | RPYNNWGQGTQVTVSSGGGGS |
| | | | EVQLVESGGGLVQAGGSLRLS |
| | | | CAASGRPFSNYAMGWFRQAPG |
| | | | KEREFVAAISWSGGSTSYADS |
| | | | VKGRFTISRDNAKNTVYLQMN |
| | | | SPKPEDTAIYYCAAQFSGADY |
| | | | GFGRLGIRGYEYDYWGQGTQV |
| | | | TVSSGLEGHSDHMEQKLISEE |
| | | | DLNRISDHHHHHH |
| 21 | GS- | Linker | GGGGS |
| | linker | | |
| 22 | V19S76 | VHH | QVQLQESGGGLVQAGGSLRLS |
| | Kt | | CAASGRTFGRSAMGWFRQAPG |
| | | | KEREFVAAIGTRGGSTKYADS |
| | | | VKGRFTISTDNAKNTVYLQMD |
| | | | SLKPEDTAVYRCAVRGPGYPS |
| | | | AAIFQDEYHYWGQGTQVTVSS |
| | | | GLEGHSDHMEQKLISEEDLNR |
| | | | ISDHHHHHH |
| 23 | V19S76 | Bispecific | QVQLQESGGGLVQAGGSLRLS |
| | K- | binding | CAASGRTFGRSAMGWFRQAPG |
| | 5C8 | molecule | KEREFVAAIGTRGGSTKYADS |
| | | | VKGRFTISTDNAKNTVYLQMD |
| | | | SLKPEDTAVYRCAVRGPGYPS |
| | | | AAIFQDEYHYWGQGTQVTVSS |
| | | | GGGGSEVQLVESGGGLVQAGG |
| | | | SLRLSCAASGRPFSNYAMGWF |
| | | | RQAPGKEREFVAAISWSGGST |
| | | | SYADSVKGRFTISRDNAKNTV |
| | | | YLQMNSPKPEDTAIYYCAAQF |
| | | | SG |
| | | | ADYGFGRLGIRGYEYDYWGQG |
| | | | TQVTVSS |
| 24 | Human | | MVRLPLQCVLWGCLLTAVHPE |
| | CD40 | | PPTACREKQYLINSQCCSLCQ |
| | | | PGQKLVSDCTEFTETECLPCG |
| | | | ESEFLDTWNRETHCHQHKYCD |
| | | | PNLGLRVQQKGTSETDTICTC |
| | | | EEGWHCTSEACESCVLHRSCS |
| | | | PGFGVKQIATGVSDTICEPCP |
| | | | VGFFSNVSSAFEKCHPWTSCE |
| | | | TKDLVVQQAGTNKTDVVCGPQ |
| | | | DRLRALVVIPIIFGILFAILL |
| | | | VLVFIKKVAKKPTNKAPHPKQ |
| | | | EPQEINFPDDLPGSNTAAPVQ |
| | | | ETLHGCQPVTQEDGKESRISV |
| | | | QERQ |
| 25 | 5C8 | Humanized | EVQLLESGGGSVQPGGSLRLS |
| | variant | sequence | CAASGRPFSNYAMSWFRQAPG |
| | | | KEREFVSAISWSGGSTSYADS |
| | | | VKGRFTISRDNSKNTLYLQMN |
| | | | SLRAEDTAVYYCAAQFSGADY |
| | | | GFGRLGIRGYEYDYWGQGTQV |
| | | | TVSS |
| 26 | 5C8 | Humanized | EVQLLESGGGLVQPGGSLRLS |
| | variant | sequence | CAASGRPFSNYAMSWFRQAPG |
| | | | KEREFVSAISWSGGSTSYADS |
| | | | VKGRFTISRDNSKNTLYLQMN |
| | | | SLRAEDTAVYYCAAQFSGADY |
| | | | GFGRLGIRGYEYDYWGQGTLV |
| | | | TVSS |
| 27 | 5C8 | Humanized | EVQLLESGGGSVQPGGSLRLS |
| | variant | sequence | CAASGRPFSNYAMSWFRQAPG |
| | | | KGLEFVSAISWSGGSTSYADS |
| | | | VKGRFTISRDNSKNTLYLQMN |
| | | | SLRAEDTAVYYCAAQFSGADY |
| | | | GFGRLGIRGYEYDYWGQGTLV |
| | | | TVSS |
| 28 | 5C8 | Humanized | EVQLLESGGGLVQPGGSLRLS |
| | variant | sequence | CAASGRPFSNYAMGWFRQAPG |
| | | | KEREFVAAISWSGGSTSYADS |
| | | | VKGRFTISRDNSKNTVYLQMN |
| | | | SLRAEDTAVYYCAAQFSGADY |
| | | | GFGRLGIRGYEYDYWGQGTLV |
| | | | TVSS |
| 29 | 5C8 | Humanized | EVQLLESGGGSVQPGGSLRLS |
| | variant | sequence | CAASGRPFSNYAMGWFRQAPG |
| | | | KEREFVAAISWSGGSTSYADS |
| | | | VKGRFTISRDNSKNTLYLQMN |
| | | | SLRAEDTAVYYCAAQ |
| | | | FSGADYGFGRLGIRGYEYDYW |
| | | | GQGTLVTVSS |
| 30 | 5C8 | Humanized | EVQLLESGGGLVQPGGSLRLS |
| | variant | sequence | CAASGRPFSNYAMGWFRQAPG |
| | | | KEREFVSAISWSGGSTSYADS |
| | | | VKGRFTISRDNSKNTVYLQMN |
| | | | SLRAEDTAVYYCAAQFSGADY |
| | | | GFGRLGIRGYEYDYWGQGTLV |
| | | | TVSS |
| 31 | 5C8 | Humanized | EVQLLESGGGSVQPGGSLRLS |
| | variant | sequence | CAASGRPFSNYAMGWFRQAPG |
| | | | KEREFVSAISWSGGSTSYADS |
| | | | VKGRFTISRDNAKNTVYLQMN |
| | | | SLRAEDTAVYYCAAQFSGADY |
| | | | GFGRLGIRGYEYDYWGQGTLV |
| | | | TVSS |
| 32 | 5C8 | Humanized | EVQLLESGGGLVQPGGSLRLS |
| | variant | sequence | CAASGRPFSNYAMGWFRQAPG |
| | | | KEREFVSAISWSGGSTSYADS |
| | | | VKGRFTISRDNAKNTVYLQMN |
| | | | SLRAEDTAVYYCAAQFSGADY |
| | | | GFGRLGIRGYEYDYWGQGTLV |
| | | | TVSS |
| 33 | 5C8 | Humanized | EVQLLESGGGLVQPGGSLRLS |
| | variant | sequence | CAASGRPFSNYAMGWFREAPG |
| | | | KEREFVSAISWSGGSTSYADS |
| | | | VKGRFTISRDNSKNTVYLQMN |
| | | | SLRAEDTAVYYCAAQFSGADY |
| | | | GFGRLGIRGYEYDYWGQGTLV |
| | | | TVSS |
| 34 | 5C8 | Humanized | EVQLLESGGGLVQPGGSLRLS |
| | variant | sequence | CAASGRPFSNYAMGWFREAPG |
| | | | KEREFVSAISWSGGSTSYADS |
| | | | VKGRFTISRDNAKNTVYLQMN |
| | | | SLRAEDTAVYYCAAQFSGADY |
| | | | GFGRLGIRGYEYDYWGQGTLV |
| | | | TVSS |
| 35 | V12 | VHH | QVQLQESGGGLVQAGGSLRLS |
| | | | CAASGLVFKRYSMNWYRQPPG |
| | | | QQRGLVASISDSGVSTNYADS |
| | | | VKGRFTISRDNAKNIGYLQMN |
| | | | SLKPEDTAVYYCNMHTFWGQG |
| | | | TQVTVSS |
| 36 | V12t | VHH | QVQLQESGGGLVQAGGSLRLS |
| | | | CAASGLVFKRYSMNWYRQPPG |
| | | | QQRGLVASISDSGVSTNYADS |
| | | | VKGRFTISRDNAKNIGYLQMN |
| | | | SLKPEDTAVYYCNMHTFWGQG |
| | | | TQVTVSSGLEGHSDHMEQKLI |
| | | | SEEDLN |
| | | | RISDHHHHHH |
| 37 | V12- | Bispecific | QVQLQESGGGLVQAGGSLRLS |
| | 5C8t | binding | CAASGLVFKRYSMNWYRQPPG |
| | | molecule | QQRGLVASISDSGVSTNYADS |
| | | | VKGRFTISRDNAKNIGYLQMN |
| | | | SLKPEDTAVYYCNMHTFWGQG |
| | | | TQVTVSSGGGGSEVQLVESGG |
| | | | GLVQAGGSLRLSCAASGRPFS |
| | | | NYAMGWFRQAPGKEREFVAAI |
| | | | SWSGGSTSYADSVKGRFTISR |
| | | | DNAKNTVYLQMNSPKPEDTAI |
| | | | YYCAAQFSGADYGFGRLGIRG |
| | | | YEYDYWGQGTQVTVSSGLEGH |
| | | | SDHMEQKLISEEDLN RISDH |
| | | | HHHHH |

Antibodies of the invention are typically produced recombinantly, i.e. by expression of nucleic acid constructs encoding the antibodies in suitable host cells, followed by purification of the produced recombinant antibody from the cell culture. Nucleic acid constructs can be produced by standard molecular biological techniques well-known in the art. The constructs are typically introduced into the host cell using an expression vector. Suitable nucleic acid constructs and expression vectors are known in the art. Host cells suitable for the recombinant expression of antibodies are well-known in the art, and include CHO, HEK-293, Expi293F, PER-C6, NS/0 and Sp2/0 cells.

According, in a further aspect, the invention relates to a nucleic acid construct encoding an antibody according to the invention, such as a multispecific antibody according to the invention. In one embodiment, the construct is a DNA construct. In another embodiment, the construct is an RNA construct.

In a further aspect, the invention relates to an expression vector comprising a nucleic acid construct an antibody according to the invention, such as a multispecific antibody according to the invention.

In a further aspect, the invention relates to a host cell comprising a nucleic acid construct encoding an antibody according to the invention, such as a multispecific antibody according to the invention or an expression vector comprising a nucleic acid construct an antibody according to the invention, such as a multispecific antibody according to the invention.

In a further aspect, the invention relates to a pharmaceutical composition comprising an antibody according to the invention, such as a multispecific antibody according to the invention, and a pharmaceutically-acceptable excipient.

Antibodies may be formulated with pharmaceutically-acceptable excipients in accordance with conventional techniques such as those disclosed in (Rowe et al., Handbook of Pharmaceutical Excipients, 2012 June, ISBN 9780857110275). The pharmaceutically-acceptable excipient as well as any other carriers, diluents or adjuvants should be suitable for the antibodies and the chosen mode of administration. Suitability for excipients and other components of pharmaceutical compositions is determined based on the lack of significant negative impact on the desired biological properties of the chosen antibody or pharmaceutical composition of the present invention (e.g., less than a substantial impact (10% or less relative inhibition, 5% or less relative inhibition, etc.) upon antigen binding). A pharmaceutical composition may include diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition. Further pharmaceutically-acceptable excipients include any and all suitable solvents, dispersion media, coatings, antibacterial and antifungal agents, isotonicity agents, antioxidants and absorption-delaying agents, and the like that are physiologically compatible with an antibody of the present invention.

In a further aspect the invention relates to the antibodies of the invention as defined herein, such as the multispecific antibodies of the invention as defined herein, for use as a medicament.

A multispecific antibody according to the invention enables creating a microenvironment that is beneficial for killing of tumor cells, in particular CD40-positive tumor cells, by Vγ9Vδ2 T cells.

Accordingly, in a further aspect the invention relates to the antibodies of the invention as defined herein, such as the multispecific antibodies of the invention as defined herein, for use in the treatment of cancer, such as chronic lymphocytic leukemia, multiple myeloma, non-Hodgkin's lymphoma, Hodgkin's lymphoma, follicular lymphoma, head and neck cancer, pancreatic cancer, ovarian cancer, lung cancer, breast cancer, colon cancer, prostate cancer, B-cell lymphoma/leukemia, Burkitt lymphoma or B acute lymphoblastic leukemia. In a preferred embodiment, the invention relates to the antibodies of the invention as defined herein, such as the multispecific antibodies of the invention as defined herein, for use in the treatment of chronic lymphocytic leukemia. In another preferred embodiment, the invention relates to the antibodies of the invention as defined herein, such as the multispecific antibodies of the invention as defined herein, for use in the treatment of multiple myeloma.

In another embodiment, the antibodies of the invention are used in the treatment of autoimmune diseases.

In some embodiments, the antibody is administered as monotherapy. However, antibodies of the present invention may also be administered in combination therapy, i.e., combined with other therapeutic agents relevant for the disease or condition to be treated. In one embodiment, the antibody is used in combination with a Bcl-2 blocker, such as venetoclax.

Similarly, in a further aspect, the invention relates to a method of treating a disease comprising administration of an antibody according to the invention, such as a multispecific antibody of the invention to a human subject in need thereof. In one embodiment, the disease is cancer.

“Treatment” or “treating” refers to the administration of an effective amount of an antibody according to the present invention with the purpose of easing, ameliorating, arresting, eradicating (curing) or preventing symptoms or disease states. An “effective amount” refers to an amount effective, at dosages and for periods of time necessary, to achieve a desired therapeutic result. An effective amount of a polypeptide, such as an antibody, may vary according to factors such as the disease stage, age, sex, and weight of the individual, and the ability of the antibody to elicit a desired response in the individual. An effective amount is also one in which any toxic or detrimental effects of the antibody are outweighed by the therapeutically beneficial effects. An exemplary, non-limiting range for an effective amount of an antibody of the present invention is about 0.1 to 100 mg/kg, such as about 0.1 to 50 mg/kg, for example about 0.1 to 20 mg/kg, such as about 0.1 to 10 mg/kg, for instance about 0.5, about 0.3, about 1, about 3, about 5, or about 8 mg/kg. Administration may be carried out by any suitable route, but will typically be parenteral, such as intravenous, intramuscular or subcutaneous.

### EXAMPLES

### Example 1: Generation of VHHs

### Introduction

Monovalent VHHs were generated that specifically bind to human CD40. These VHHs were then used to generate bispecific anti-CD40-anti-Vγ9Vδ2 TCR VHHs.

### Material and Methods

### Generation of Monovalent Vγ9Vδ2-TCR Specific VHHs

The Vγ9Vδ2-TCR specific VHH 5C8 (SEQ ID NO:17), binding to the V52 chain of the Vγ9Vδ2-T cell receptor, was previously generated (de Bruin et al. (2016), Clin Immunol 169:128-138) (WO2015156673).

### Generation of Monovalent CD40-Specific VHHs

### Lama Immunization

CD40-specific VHHs were generated as previously described (de Bruin et al. (2016), Clin Immunol 169:128-138, Lameris et al. (2016), Immunology 149(1)111-21). Two lamas (llama glama) were immunized six times with 50*10⁶ MUTZ-3 DC (see e.g. Masterson (2002) Blood 100:701) cells with a one-week interval.

### Construction of VHH Phage Library

RNA was isolated from peripheral blood lymphocytes obtained 1 week after the last immunization, transcribed into cDNA and used for Ig-heavy chain-encoding gene amplification (Roovers et al. (2007) Cancer Immunol Immunother 56(3):303-317). Phage libraries were constructed by ligation of VHH-encoding genes into the phagemid vector pUR8100 containing a Myc- and His6-tag encoding fragment and subsequent transformation into E. coli TG1 for display on filamentous bacteriophage.

### Enrichment and Selection of CD40-Specific VHH

To enrich for phages displaying CD40-specific VHHs, multiple selection rounds were performed. Plates were coated with IgG1-Fc-tagged human CD40 (71174, BPS Bioscience, San Diego, Calif., USA). Phages were blocked with PBS containing 1% bovine serum albumin, 1% milk, 0.05% Tween 20 and human IgG (0.625 mg/mL) and then allowed to bind to the CD40-coated plates. Eluted phages were used to infect exponentially growing E. coli TG1.

After two such rounds, ELISA-based screening was performed to select for binding to human CD40, but not human Ig. For this purpose, plates were coated either with IgG1-Fc-tagged human CD40 or human Ig and incubated with periplasmic extracts from the transformed TG1. Bound extracts were detected by sequential incubation with mouse-derived anti-Myc tag (05-274, Merck, Kenilworth, N.J., USA) and HRP-conjugated rabbit-derived anti-mouse IgG antibodies. DNA sequence analysis of selected clones demonstrated three different CD40-specific VHH sequences. The encoded amino acid sequences are shown in the sequence listing herein. SEQ ID NO:13 shows the V19 VHH sequence, SEQ ID NO:14 shows the V15 VHH sequence and SEQ ID NO:35 shows the V12 VHH sequence.

### VHH Production and Purification

Gene segments encoding the three selected monovalent VHHs and a Myc- and His6-tag were re-cloned into the pcDNA5 vector, which was used to transfect HEK293T cells. VHH protein was purified from the HEK293T supernatant by sequential size exclusion, Ni-based His-tag selection and imidazole-based elution using fast protein liquid chromatography. The three different VHH proteins were termed V19t (SEQ ID NO:15), V15t (SEQ ID NO:16) and V12t (SEQ ID NO:36), wherein ‘t’ indicates that the VHH contains a C-terminal Myc- and His6-tag. VHH integrity and purity was confirmed by Coomassie blue staining in SDS-PAGE gels and western blotting using anti-Myc tag antibodies. VHH was quantified using a Nanodrop spectrophotometer.

### Generation of Bispecific Constructs

To generate bispecific VHH constructs V19-5C8t (SEQ ID NO:19), V15-5C8t (SEQ ID NO:20) and V12-5C8t (SEQ ID NO:37), the anti-Vδ2-TCR-VHH (C-terminal) (SEQ ID NO: 17) was joined to the anti-CD40-VHHs (N-terminal) with a Gly4Ser-linker (SEQ ID NO:21). The bispecific VHHs, containing a Myc- and His6-tag, were produced by HEK293T transfection as described above. VHH protein was purified from the supernatant using immobilized ion affinity chromatography on Talon resin (635503, Clontech, Mountain View, Calif., USA) followed by imidazole-based elution.

### Generation of V19S76K-5C8

A putative glycosylation site in framework region 3 of the V19t VHH was identified, after which a new VHH (V19S76Kt) (SEQ ID NO:22) was produced and purified in which the relevant serine (position 76) was altered into a lysine. The bispecific V19S76K-5C8t VHH was constructed as described above. Tag-less V19S76K (SEQ ID NO:23) was generated as described above by UPE (Utrecht, the Netherlands).

### Example 2: Monovalent VHH Binds to CD40-Transfected Cells

### Introduction

The ability of the monovalent anti-CD40 VHH to bind specifically to CD40-expressing cells was tested.

### Materials and Methods

### Cell Lines

The embryonic kidney cell line HEK293T, either wildtype (WT) or transfected with human CD40, was grown in Dulbecco's Modified Eagle Medium (41965-039, Thermo Fisher Scientific, Waltham, Mass., USA), supplemented with 10% fetal calf serum (F7524, Merck, Kenilworth, N.J., USA), 200 mM L-glutamine (25030-123, Thermo Fisher Scientific), 0.05 mM β-mercapto-ethanol (M6250, Merck) and 10,000 U/mL penicillin/streptomycin (15140-122, Thermo Fisher Scientific), hereafter referred to as complete DMEM.

### VHH Binding

CD40 expression on CD40-transfected cells was confirmed by incubation with a PE-conjugated anti-CD40 antibody (IM1936U, Beckman Coulter, Brea, Calif., USA) for 20 minutes at 4° C. To assess VHH binding, cells were incubated with 100 nM V15t, 100 nM V19t or medium control for 30 minutes at 37° C. Bound VHH was detected by sequential incubation with mouse-anti-Myc tag (05-274, Merck) and AF488-conjugated goat-anti-mouse (A-11001, Thermo Fisher Scientific) antibodies for 20 minutes at 4° C.

### Flow Cytometry

Samples were measured on a FACSCanto cytometer (BD Biosciences, Franklin Lakes, N.J., USA) and analyzed with Flowjo MacV10.

### Results

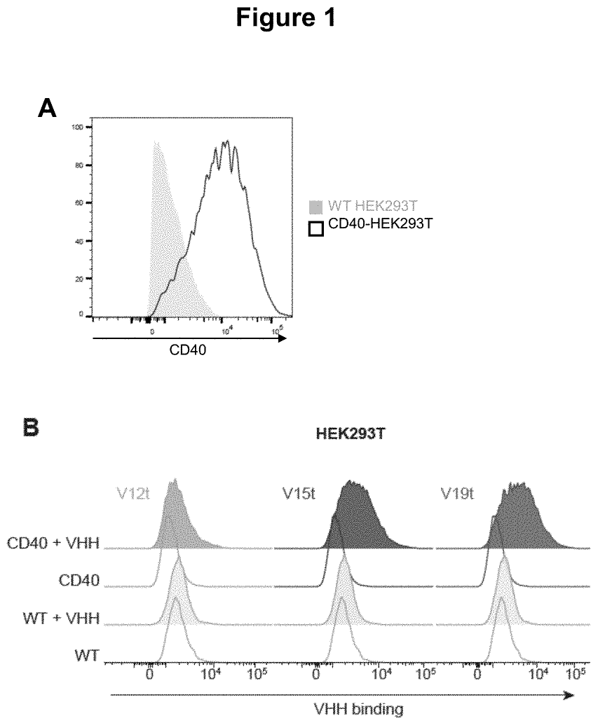

WT and CD40-transfected HEK293T cells were used to test the binding of the monovalent anti-CD40 VHH. CD40 expression was confirmed on the CD40-transfected cells (FIG. 1A). V19t, V15t and V12t bound to the CD40-expressing cells, as demonstrated by detection of the Myc tag (FIG. 1B). In contrast, the anti-CD40 VHHs did not bind to the CD40-negative WT HEK293T cells.

Furthermore, mutation of glycosylation site in V19t (S76K mutation) did not impair binding capacity to CD40, see Table 1.

TABLE 1
binding of V19t and V19S76Kt to CD40-expressing cells

| VHH | | | |
|---|---|---|---|
| concentration | | V19t | V19S76Kt |
| 0 | pM | 648 | 648 |
| 1 | pM | 5031 | 4790 |
| 10 | pM | 4938 | 4949 |
| 100 | pM | 5538 | 5502 |
| 1 | nM | 6783 | 7906 |
| 10 | nM | 9101 | 9283 |
| 100 | nM | 16175 | 17062 |

Table 1: Mutation of glycosylation site in V19t does not impair binding capacity to CD40. CD40-transfected HEK293T cells were incubated with the indicated concentrations of V19t or V19S76Kt and the Myc-tag was subsequently detected by flow cytometry. The average geometric mean fluorescence intensity obtained in 2 experiments is shown.

### Conclusion

The anti-CD40 VHHs V19t and V15t bind specifically to cell surface-expressed CD40 and the binding affinity of V19t was retained in V19S76Kt.

### Example 3: Monovalent VHH Binds to Primary CLL Cells

### Introduction

Primary chronic lymphocytic leukemia (CLL) cells express CD40 on the cell surface. Thus, the binding of the anti-CD40 VHH to primary CLL cells was tested.

### Materials and Methods

### Patient Material

Peripheral blood (PB) mononuclear cells (PBMCs, ≥95% CD5⁺CD19⁺) were isolated from PB samples from untreated CLL patients and cryopreserved as described previously (Hallaert et al. (2008), Blood 112(13):5141-9). The study was approved by the medical ethics committee at the Amsterdam UMC. Written informed consent from all subjects was obtained. Thawed cells were kept in Iscove's Modified Dulbecco's Medium (IMDM; 12440-053, Thermo Fisher Scientific), supplemented with 10% fetal calf serum (F7524, Merck), 200 mM L-glutamine (25030-123, Thermo Fisher Scientific), 0.05 mM β-mercapto-ethanol (M6250, Merck) and 10.000 U/mL penicillin/streptomycin (15140-122, Thermo Fisher Scientific), hereafter referred to as complete IMDM.

### VHH Binding and Flow Cytometry

CD40 expression on primary CLL cells was confirmed and VHH binding was tested as described in Example 2.

### Results

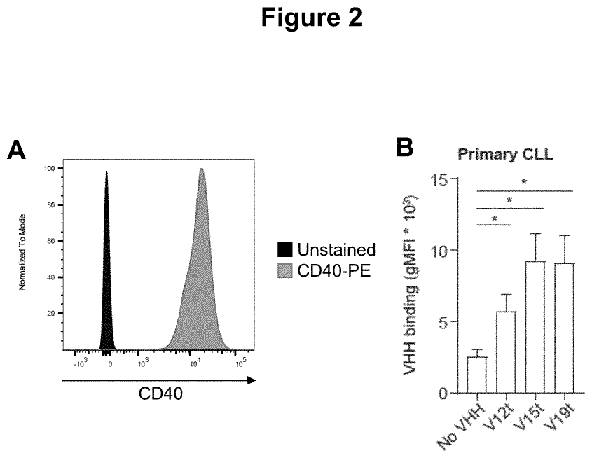

Primary CLL cells homogenously expressed CD40 (FIG. 2A). The anti-CD40 VHHs evidently bound to primary CLL cells in all samples tested, although V15t and V19t had a higher binding intensity than V12t (FIG. 2B).

### Conclusion

The anti-CD40 VHHs bind to primary CLL cells.

### Example 4: Monovalent VHH is not a CD40 Agonist

### Introduction

Binding of CD40 to its cognate ligand CD40L can lead to a variety of biological responses. The effects induced by CD40 stimulation in primary CLL cells include cellular growth and an increased expression of costimulatory molecules (i.e. CD86) and the Fas receptor (CD95). The capacity of the anti-CD40 VHH to induce CD40 stimulation was tested in primary CLL cells.

### Materials and Methods

### Patient Material

PBMCs (≥90% CD5⁺CD19⁺) from untreated CLL patients were obtained and cryopreserved as described in Example 3. Thawed cells were kept in complete IMDM.

### Agonistic Activity

To assess whether binding of the VHH to CD40 has agonistic effects, primary CLL PBMCs were cultured for 48 hours in the presence of VHH, medium control or recombinant multimeric CD40 ligand (rmCD40L; 100 ng/mL, Bioconnect).

### Flow Cytometry

After 48 hours, cells were harvested, washed and incubated with AF700-conjugated anti-CD19 (557921), FITC-conjugated anti-CD80 (6109965), APC-conjugated anti-CD86 (555660, all BD Biosciences), PE-conjugated anti-CD5 (12-0059-42, Thermo Fisher Scientific) and PECy7-conjugated anti-CD95 (305621, Biolegend, San Diego, Calif., USA) antibodies for 20 minutes at 4° C. Alternatively, after 48 hours, cells were harvested and viability was measured using Mitotracker Orange (25-minute incubation at 37° C.) and To-pro-3 (10-minute incubation at room temperature; both Thermo Fisher Scientific). Samples were measured on a FACSCanto cytometer (BD Biosciences) and analyzed with Flowjo MacV10.

### Results

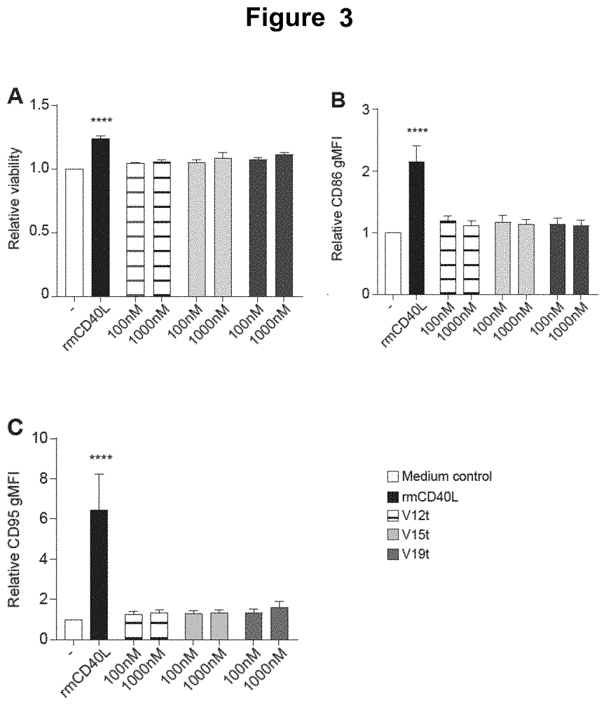

rmCD40L effectively induced CD40 stimulation, as demonstrated by an increase in viability and expression of CD86 and CD95 (FIG. 3A-C). The anti-CD40 VHHs V19t, V15t and V12t on the other hand did not induce any of these effects in the various concentrations tested.

### Conclusion

The monovalent anti-CD40 VHHs are not agonists of CD40.

### Example 5: Monovalent VHH Antagonizes CD40 Stimulation

### Introduction

CD40L binding can induce CD40 stimulation. Since both CD40L and the anti-CD40 VHH can bind CD40, it was tested whether the anti-CD40 VHH could prevent CD40L-induced CD40 stimulation.

### Materials and Methods

### Patient Material

PBMCs (≥90% CD5⁺CD19⁺) from untreated CLL patients were obtained and cryopreserved as described in Example 2. Thawed cells were kept in complete IMDM.

### Antagonistic Activity

To test whether the VHH antagonizes CD40 stimulation, primary CLL PBMCs were pre-incubated with VHH or medium control for 30 minutes at 37° C. and subsequently cultured for 48 hours in the presence of rmCD40L (100 ng/mL).

### Flow Cytometry

After 48 hours, cells were analyzed by flow cytometry as described in Example 4.

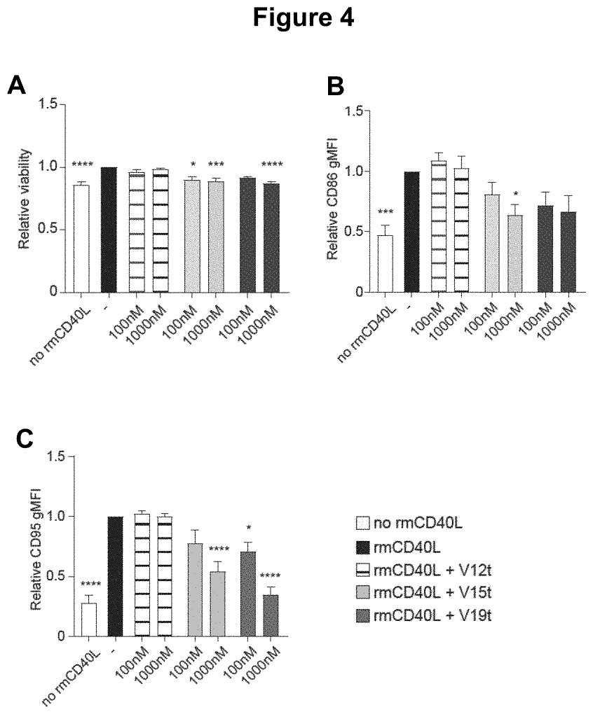

Results rmCD40L effectively induced CD40 stimulation, as demonstrated by an increase in viability and expression of CD86 and CD95 (FIG. 4A-C). Pre-incubation with either V15t or V19t prevented CD40 stimulation in a dose-dependent manner.

However, V12t did not block CD40L-induced effects.

Conclusion

The monovalent anti-CD40 VHHs V15t and V19t antagonize CD40 stimulation.

### Example 6: Bispecific VHH Antibody Binds CD40-Transfected Cells

### Introduction

The ability of the bispecific anti-CD40-anti-Vγ9Vδ2-TCR VHH construct V19S76K-5C8 to bind specifically to CD40-expressing cells was tested.

### Materials and Methods

### VHH Generation

The bispecific anti-CD40-anti-Vγ9Vδ2-TCR VHH V19S76K-5C8 was generated as described in Example 1.

### Cell Line

The embryonic kidney cell line HEK293T, either wildtype (WT) or transfected with human CD40, was grown in complete DMEM.

### VHH Binding

To assess VHH binding, cells were incubated with V19S76K-5C8 (1 μM) or medium control for 30 minutes at 37° C. Bound VHH was detected by incubation with FITC-conjugated goat-anti-llama IgG-heavy and light chain antibodies (A160-100F, Bethyl Laboratories Inc., Montgomery, Tex., USA) for 20 minutes at 4° C.

### Flow Cytometry

After 48 hours, cells were analyzed by flow cytometry as described in Example 2.

### Results

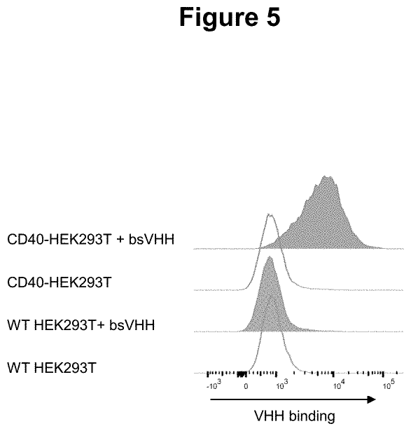

V19S76K-5C8 binds to the CD40-expressing HEK293T cells, but not to CD40-negative WT HEK293T cells (FIG. 5).

### Conclusion

The bispecific anti-CD40-anti-Vγ9Vδ2 TCR VHH V19S76K-5C8 binds specifically to cell surface-expressed CD40.

### Example 7: Bispecific VHH Antibody Binds CD40⁺ and Vγ9Vδ2⁺ Cells

### Introduction

The ability of the bispecific anti-CD40-anti-Vγ9Vδ2 TCR VHH V19S76K-5C8 to bind to CD40⁺ and Vγ9Vδ2⁺ cells was tested.

### Materials and Methods

### Cell Lines

The CLL-derived cell line CII was grown in complete IMDM. Purified Vγ9Vδ2-T cell lines were generated as described previously (de Bruin et al. (2017), Oncoimmunology 7(1): e1375641). In short, Vδ2⁺-T cells were isolated from healthy donor (HD) PBMCs using FITC-conjugated anti-Vδ2 TCR (2257030, Sony, San Jose, Calif.) in combination with anti-mouse IgG microbeads (Miltenyi Biotec, Bergisch Gladbach, Germany) and cultured weekly with irradiated feeder mix consisting of PBMCs from 2 HDs, JY cells, IL-7 (10 U/mL), IL-15 (10 ng/mL, R&D Systems) and phytohaemagglutinin (PHA; R30852801, Thermo Fisher Scientific).

Purity of Vγ9Vδ2-T cell lines was maintained at >90%.

### VHH Binding

VHH binding was tested as described in Example 6.

### Flow Cytometry

After 48 hours, cells were analyzed by flow cytometry as described in Example 2.

### Results

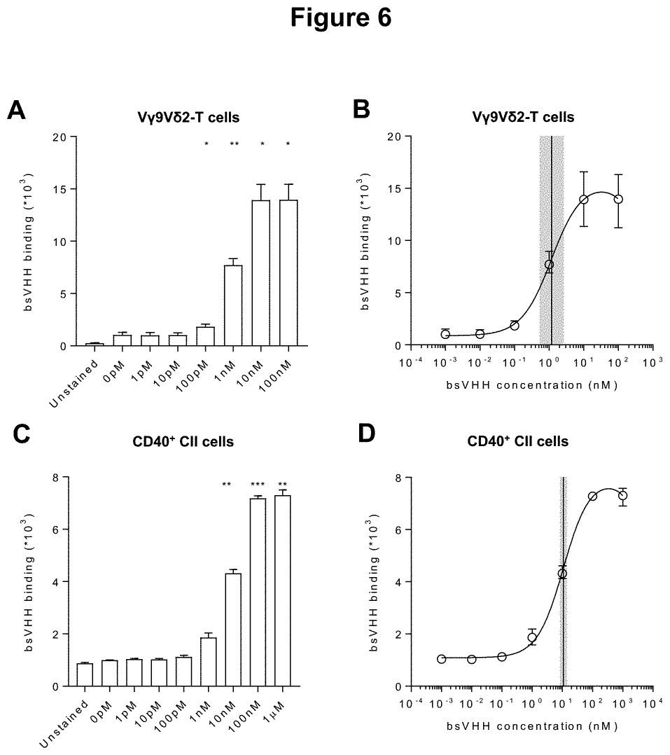

V19S76K-5C8 binds to Vγ9Vδ2⁺ cells with an apparent Kd of 1.2 nM (FIGS. 6A and B). Likewise, V19S76K-5C8 binds to CD40⁺ CII cells with an apparent Kd of 10.9 nM as determined by flowcytometry (FIGS. 6C and D).

### Conclusion

The bispecific anti-CD40-anti-Vγ9Vδ2 TCR VHH V19S76K-5C8 binds to both CD40⁺ and Vγ9Vδ2⁺ cells.

### Example 8: Bispecific VHH Antibody is not a CD40 Agonist

### Introduction

The monovalent anti-CD40 VHH V19t does not induce CD40 stimulation. Whether CD40 stimulation also does not occur when V19 is incorporated in the bispecific VHH V19S76K-5C8 was tested using primary CLL cells.

### Materials and Methods

### Patient Material, Agonistic Activity and Flow Cytometry

To assess whether binding of the VHH has agonistic effects, primary CLL PBMCs were cultured with the indicated concentrations of V19S76K-5C8, medium control or rmCD40L for 48 hours and analyzed by flow cytometry as described in Example 4.

### Results

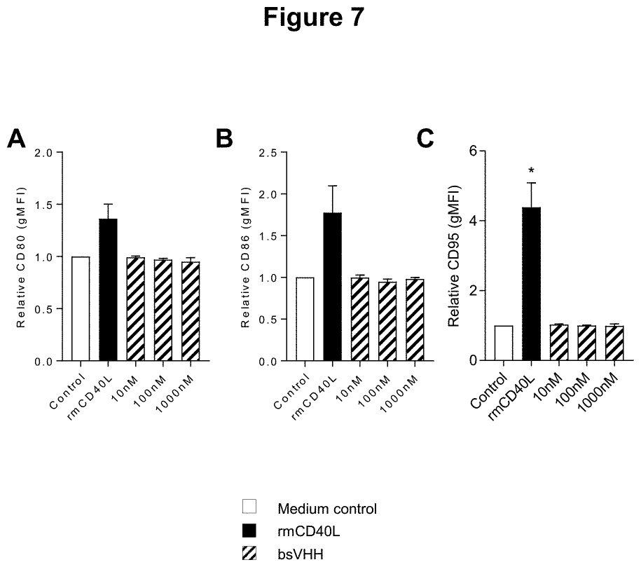

rmCD40L effectively induced CD40 stimulation, as demonstrated by an increase in expression of CD80, CD86 and CD95 (FIG. 7A-C). On the contrary, none of the V19S76K-5C8 concentrations tested increased the expression of CD80, CD86 or CD95.

### Conclusion

The bispecific anti-CD40-anti-Vγ9Vδ2 TCR VHH V19S76K-5C8 is not an agonist of CD40.

### Example 9: Bispecific VHH Antibody Antagonizes CD40 Stimulation

### Introduction

The monovalent anti-CD40 VHH V19t prevents the effects induced by CD40L-induced CD40 stimulation. Whether the CD40 antagonistic activity is retained in the bispecific V19S76K-5C8 format was tested using primary CLL cells.

### Materials and Methods

### Patient Material, Antagonistic Activity and Flow Cytometry

To assess whether binding of the VHH has antagonistic effects, primary CLL PBMCs were pre-incubated with V19S76K-5C8 or medium control and then cultured with rmCD40L for 48 hours and analyzed by flow cytometry as described in Example 5.

### Results

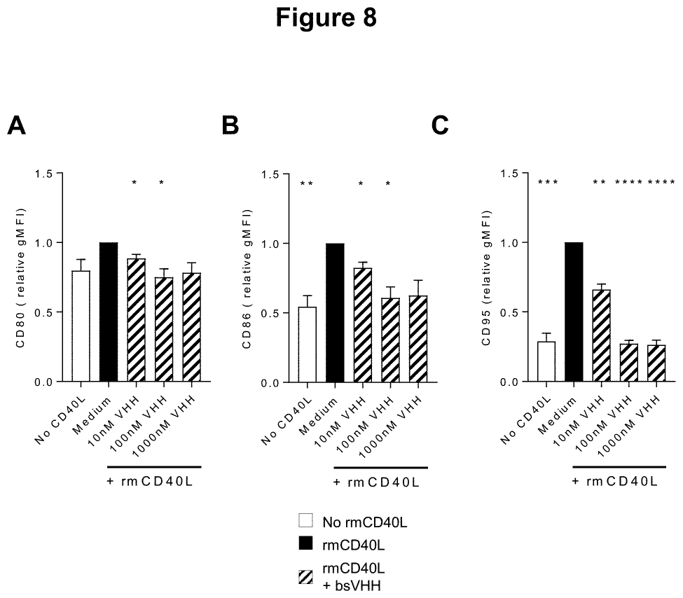

rmCD40L led to a higher expression of CD80, CD86 and CD95, indicating CD40 stimulation (FIG. 8A-C). Pre-incubation with V19S76K-5C8 prevented the effects of CD40 stimulation in a dose-dependent manner.

### Conclusion

The bispecific anti-CD40-anti-Vγ9Vδ2 TCR VHH V19S76K-5C8 retains antagonistic CD40 activity.

### Example 10: Bispecific VHH Antibody Sensitizes Primary CLL Cells to Venetoclax

### Introduction

CD40 stimulation leads to resistance of primary CLL cells towards venetoclax (ABT-199), an inhibitor of the anti-apoptotic protein Bcl-2 (Thijssen et al. (2015), Haematologica 100(8):e302-6). This is presumably caused by an upregulation of the anti-apoptotic protein Bcl-xL. Since V19S76K-5C8 antagonizes CD40 stimulation, the capacity of V19S76K-5C8 to reverse the CD40-induced venetoclax resistance was tested.

### Materials and Methods

### Patient Material, Antagonistic Activity and Venetoclax Sensitivity

Primary CLL PBMCs were pre-incubated with V19S76K-5C8 (1000 nM) or medium control and then cultured with rmCD40L for 48 hours and analyzed by flow cytometry as described in Example 8. Cytofix/Cytoperm reagent (554722, BD Biosciences) was used for detection of intracellular Bcl-xL (13835S, Cell Signaling, Danvers, Mass., USA). After 48 hours, cells were cultured with the indicated concentrations of venetoclax (Bioconnect, Huissen, the Netherlands) for 24 hours.

### Viability Measurement and Flow Cytometry

Viability was measured as described in Example 4 Cells were analyzed by flow cytometry as described in Example 2.

### Results

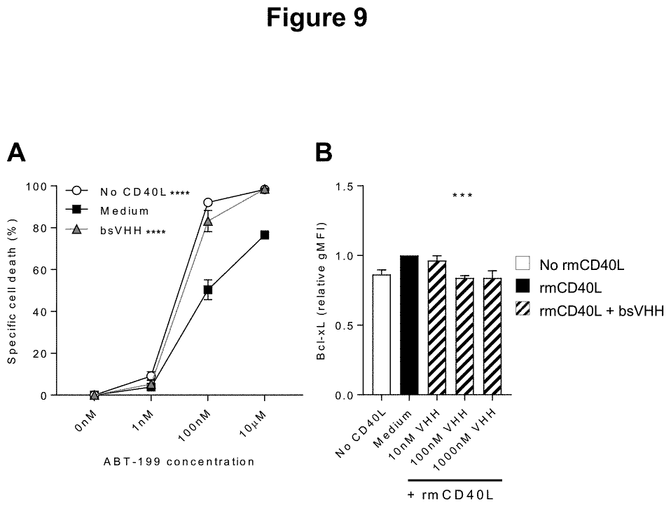

Venetoclax induced cell death in unstimulated primary CLL cells in a dose-dependent manner (FIG. 9A). Primary CLL cells that were stimulated with rmCD40L were less sensitive to venetoclax. However, cells that were cultured with V19S76K-5C8 in addition to rmCD40L were as sensitive to venetoclax as unstimulated CLL cells. This correlated with Bcl-xL expression, which increased upon rmCD40L stimulation, but returned to unstimulated levels when rmCD40L was preceded by V19S76K-5C8 incubation (FIG. 9B).

### Conclusion

The bispecific anti-CD40-anti-Vγ9Vδ2 TCR VHH V19S76K-5C8 sensitizes primary CLL cells towards venetoclax.

### Example 11: Bispecific VHH Antibody Activates Vγ9Vδ2-T Cells

### Introduction

The bispecific anti-CD40-anti-Vγ9Vδ2 TCR VHH V19S76K-5C8 can bind both CD40 on target cells and the Vγ9Vδ2-T cell receptor. The ability of V19S76K-5C8 to activate Vγ9Vδ2-T cells in the presence of CD40⁺ cells was tested.

### Materials and Methods

### Cell Lines

CD40⁺ CII cells and Vγ9Vδ2-T cells were grown as described in Example 7.

### Cytokine and Degranulation Assay

Vγ9Vδ2-T cell lines were incubated with V19S76K-5C8 or medium control for 30 minutes at 37° C. Subsequently, Vγ9Vδ2-T cells were cocultured with CII cells for 4 hours in a 1:1 ratio in the presence of Brefeldin A (10 μg/mL; B7651, Merck), GolgiStop (554724) and PECy7-conjugated anti-CD107a (561348, both BD Biosciences). Cells were then washed and surface staining was performed with Fixable Viability Dye eFluor506 (65-0866-14), AF700-conjugated anti-CD3 (56-0038-82, both Thermo Fisher Scientific) and FITC-conjugated anti-Vγ9-TCR (IM1463, Beckman Coulter) antibodies. Cytofix/Cytoperm reagent (554722) was used for detection of intracellular cytokines with BUV395-conjugated anti-IFN-γ (563563), BVδ50-conjugated anti-TNF-α (563418, all BD Biosciences) and PE/Dazzle594-conjugated anti-IL-2 (500343, Biolegend).

### Flow Cytometry

Samples were measured on an LSRFortessa cytometer (BD Biosciences) and analyzed with Flowjo MacV10.

### Results

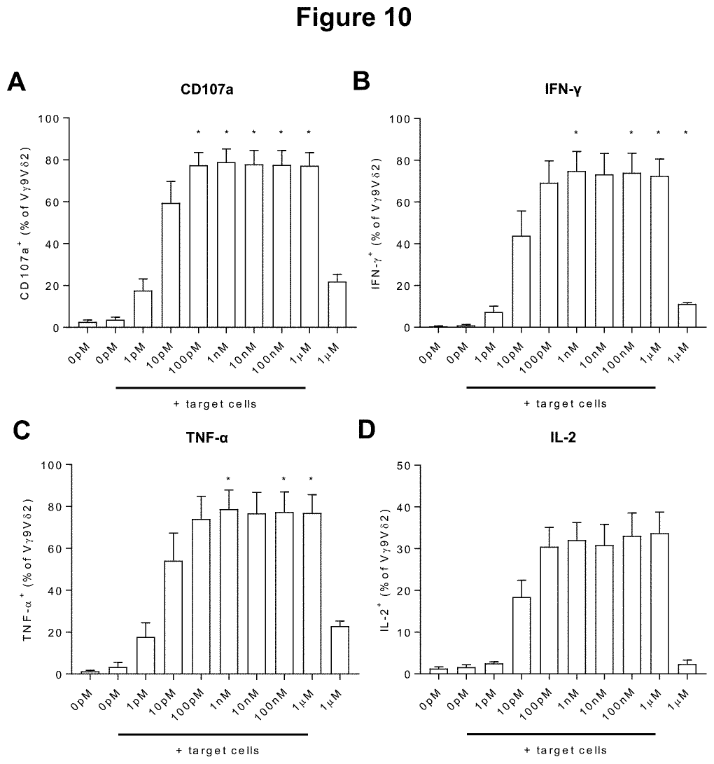

Vγ9Vδ2-T cells hardly degranulated when cultured alone or with CII cells (FIG. 10A). However, when both V19S76K-5C8 and CD40⁺ CII cells were present the large majority of Vγ9Vδ2-T cells degranulated. V19S76K-5C8 did not induce this level of degranulation when CD40⁺ CII cells were not present. A similar pattern was observed for IFN-γ, TNF-α and IL-2 production (FIG. 10B-D).

### Conclusion

The bispecific anti-CD40-anti-Vγ9Vδ2 TCR VHH V19S76K-5C8 activates Vγ9Vδ2-T cells in the presence of CD40⁺ cells.

### Example 12: Bispecific VHH Antibodies Enhances Cytotoxicity Against CD40⁺ Cells

### Introduction

The bispecific anti-CD40-anti-Vγ9Vδ2 TCR VHHs V15-5C8t and V19-5C8 bind both CD40 and Vγ9Vδ2-T cells. Whether the bispecific VHHs also induce cytotoxicity towards CD40⁺ target cells was tested.

### Materials and Methods

### VHH Generation

The bispecific V15-5C8t and V19S76K-5C8 VHHs, were generated as described in Example 1.

### Cell Lines

CD40⁺ CII cells and Vγ9Vδ2-T cells were grown as described in Example 7.

### Cytotoxicity Assay

CII target cells were labeled with carboxyfluorescein succinimidyl ester (CFSE; C1157, Thermo Fisher Scientific) and incubated with VHH or medium control for 30 minutes at 37° C. Target cells were then cocultured overnight with Vγ9Vδ2-T cell lines in a 1:1 ratio.

### Viability Measurement and Flow Cytometry

Viability was measured as described in Example 4.

### Results

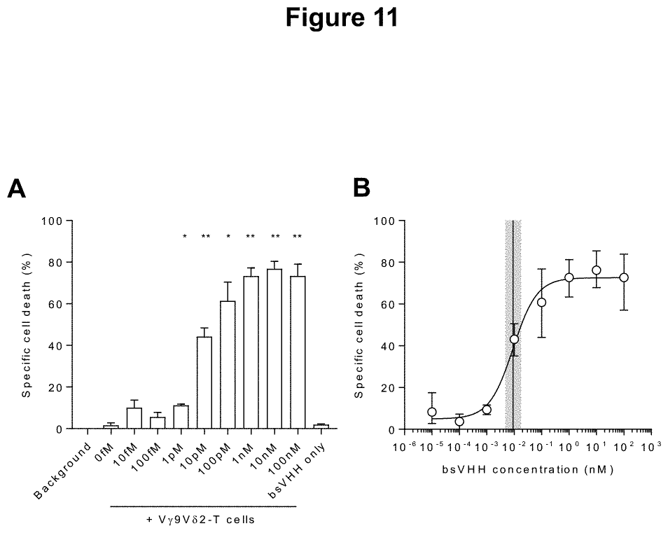

Vγ9Vδ2-T cells lysed only a minority of CII target cells (FIG. 11A). The lysis of CII target cells increased markedly when V19S76K-5C8 was added, in a dose-dependent manner. Similar results were obtained with V15-5C8t and V12-5C8t, although V12-5C8t was less potent (data not shown). The half maximal effective concentration for V19S76K-5C8 was 9.1 μM (FIG. 11B).

### Conclusion

The bispecific anti-CD40-anti-Vγ9Vδ2 TCR VHHs enhance cytotoxicity towards CD40⁺ cells.

### Example 13: Bispecific VHH Cytotoxicity is CD40 Specific

### Introduction

The bispecific anti-CD40-anti-Vγ9Vδ2 TCR VHH V19S76K-5C8 increases the cytotoxicity towards CD40⁺ target cells. The specificity towards CD40 of the enhanced cytotoxicity was tested.

### Materials and Methods

### Cell Lines

HEK293T cells, either wildtype (WT) or transfected with human CD40, were grown as described in Example 2. Vγ9Vδ2-T cells were grown as described in Example 7.

### Cytotoxicity Assay

The cytotoxicity experiment, viability measurement and flow cytometry were performed as described in Example 12.

### Results

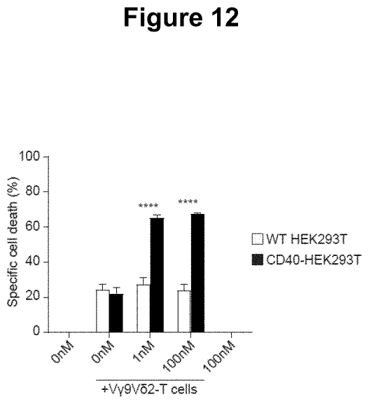

Vγ9Vδ2-T cells lysed approximately 20% of both the WT and the CD40-transfected HEK293T cells (FIG. 12). Addition of V19S76K-5C8 strongly enhanced the lysis of CD40-transfected HEK293T cells, but not of CD40-negative WT HEK293T cells. V19S76K-5C8 did not induce lysis of either WT or CD40-transfected HEK293T cells without Vγ9Vδ2-T cells.

### Conclusion

The bispecific anti-CD40-anti-Vγ9Vδ2 TCR VHH V19S76K-5C8 enhances cytotoxicity in a CD40-specific manner.

### Example 14: Bispecific VHH Antibodies Enhance Cytotoxicity Against Primary CLL Cells

### Introduction

The bispecific anti-CD40-anti-Vγ9Vδ2 TCR VHHs V15-5C8t, V19-5C8t and V12-5C8t enhance cytotoxicity of CD40⁺ target cells and now the effect on cytotoxicity towards primary CLL cells was assessed.

### Materials and Methods

### Patient Material and Cell Lines

Primary CLL cells were obtained, cryopreserved and thawed as described in Example 3. Vγ9Vδ2-T cells were grown as described in Example 7.

### Cytotoxicity Assay

The cytotoxicity experiment, viability measurement and flow cytometry were performed as described in Example 12.

### Results

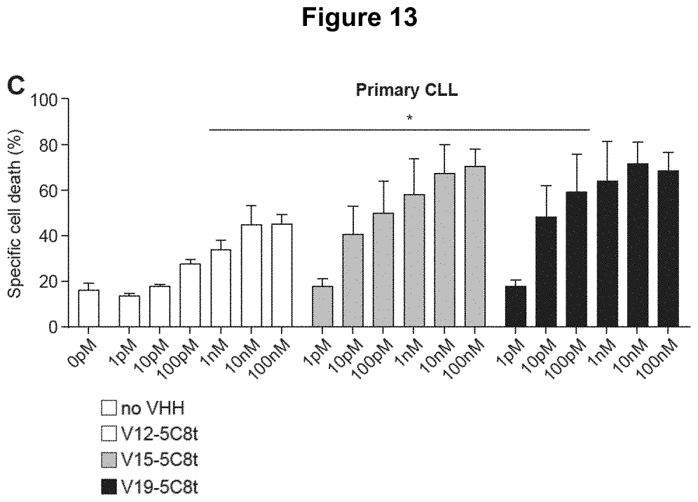

Vγ9Vδ2-T cells lysed a minority of primary CLL cells (FIG. 13), which was clearly enhanced by V12-5C8t (100 nM; 45.3%±4.0), and in particular by V15-5C8t (70.5%±7.3) and V19-5C8t (68.5%±7.9).

### Conclusion

The bispecific anti-CD40-anti-Vγ9Vδ2 TCR VHHs enhance cytotoxicity towards primary CLL cells.

### Example 15: Bispecific VHH Antibody is Effective Against CD40-Stimulated CLL Cells

### Introduction

The bispecific anti-CD40-anti-Vγ9Vδ2 TCR VHH V19S76K-5C8 increases the cytotoxicity towards primary CLL cells. CD40 stimulation increases the resistance of primary CLL cells towards various drugs, such as venetoclax (ABT-199; Thijssen et al. (2015), Haematologica 100(8):e302-6). Thus, the sensitivity of CD40-stimulated primary CLL cells to V19S76K-5C8-induced cytotoxicity was assessed.

### Materials and Methods

### Patient Material and Cell Lines

Primary CLL cells were obtained, cryopreserved and thawed as described in Example 3. 3T3 fibroblasts, either WT or transfected with human CD40L (3T40L), were grown in complete IMDM. Vγ9Vδ2-T cells were grown as described in Example 7.

### CD40 Stimulation

Primary CLL cells were cultured for 72 hours on irradiated 3T3 or 3T40L fibroblasts to induce CD40 stimulation.

### Cytotoxicity Assay

Cells were then harvested and cultured overnight either with venetoclax (10 nM) as described in Example 10, or with Vγ9Vδ2-T cells and V19S76K-5C8 as described in Example 12. Viability measurement and flow cytometry were performed as described in Example 10.

### Results

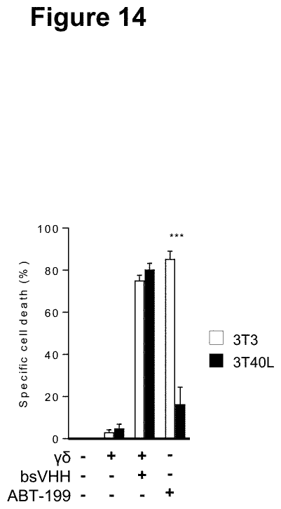

Venetoclax induced cell death in the majority of unstimulated CLL cells, but 3T40L-induced CD40 stimulation increased the resistance of CLL cells towards venetoclax (FIG. 14). In contrast, V19S76K-5C8 induced cytotoxicity in unstimulated and CD40-stimulated CLL cells to a similar extent.

### Conclusion

The bispecific anti-CD40-anti-Vγ9Vδ2 TCR VHH V19S76K-5C8 is effective against CD40-stimulated CLL cells.

### Example 16: Bispecific VHH Antibody Activates Autologous Vγ9Vδ2-T Cells from CLL Patients

### Introduction

The bispecific anti-CD40-anti-Vγ9Vδ2 TCR VHH V19S76K-5C8 activates Vγ9Vδ2-T cell lines when CD40⁺ cells are present. The ability of V19S76K-5C8 to activate Vγ9Vδ2-T from CLL patients in the presence of their own CLL cells was tested.

### Materials and Methods

### Patient Material

PBMCs from CLL patients were obtained, cryopreserved and thawed as described in Example 3.

### Cytokine and Degranulation Assay

CLL PBMCs were partially depleted of CD19⁺ CLL cells using magnetic beads (130-050-301, Miltenyi Biotec. ±50% of the PBMCs were CD19⁺ after CD19 depletion). PBMCs were then cultured overnight with V19S76K-5C8 (10 nM) or medium control in the presence of Brefeldin A, GolgiStop and anti-CD107a to measure cytokine production and degranulation as described in Example 11. In contrast to Example 11, surface staining included PE-conjugated anti-Vγ9-TCR (2256535, Sony) and FITC-conjugated goat-anti-Ilama IgG-heavy and light chain antibodies (A160-100F, Bethyl Laboratories Inc.)

### Results

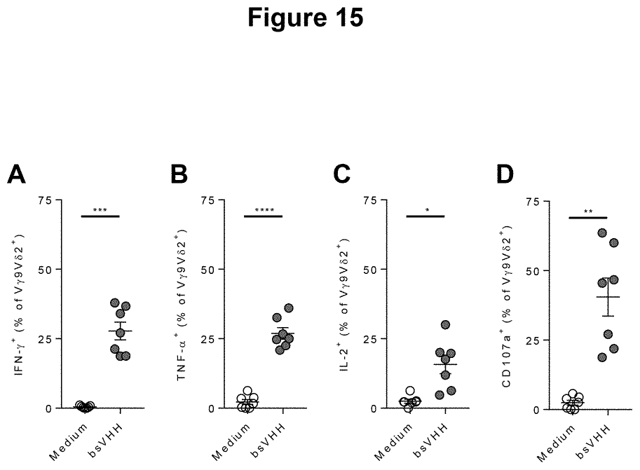

Vγ9Vδ2-T cells from CLL patients produced the cytokines IFN-γ (FIG. 15A), TNF-α (FIG. 15B) and IL-2 (FIG. 15C) after culture with V19S76K-5C8. Likewise, V19S76K-5C8 induced Vγ9Vδ2-T cell degranulation, as measured by CD107a expression (FIG. 15D).

### Conclusion

The bispecific anti-CD40-anti-Vγ9Vδ2 TCR VHH V19S76K-5C8 activates autologous Vγ9Vδ2-T cells from CLL patients.

### Example 17: Bispecific VHH Antibody Induces Cytotoxicity of CLL Cells by Autologous Vγ9Vδ2-T Cells

### Introduction

The bispecific anti-CD40-anti-Vγ9Vδ2 TCR VHH V19S76K-5C8 activates autologous Vγ9Vδ2-T cells from CLL patients. Whether this also leads to lysis of autologous CLL cells was determined.

### Materials and Methods

### Patient Material

PBMCs from CLL patients were obtained, cryopreserved and thawed as described in Example 3.

### Cytotoxicity Assay

CD3⁺ cells were isolated from CLL PBMCs using magnetic beads (purity ≥93%; 130-050-101, Miltenyi Biotec) to simultaneously enrich for Vγ9Vδ2-T cells. CD19⁺ CLL cells were isolated from the same sample using magnetic beads (purity ≥93%; 130-050-301, Miltenyi Biotec). CD3⁺ cells were cultured overnight with CD19⁺ CLL cells in a 10:1 ratio with V19S76K-5C8 (10 nM) or medium control.

### Flow Cytometry

Samples were incubated with Fixable Viability Dye eF780 (65-0865-14), PerCPeF710-conjugated anti-CD3), PE-conjugated anti-CD5 (12-0059-42, all Thermo Fisher Scientific) and FITC-conjugated anti-CD20 (A07772, Beckman Coulter) antibodies. Live CLL cells were then quantified using counting beads (01-1234-42, Thermo Fisher Scientific) on a FACSCanto cytometer (BD Biosciences).

### Results

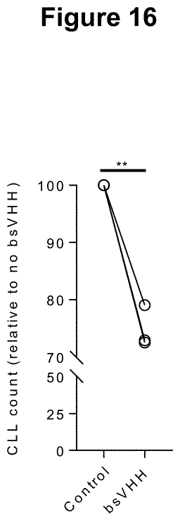

Fewer CLL cells were alive after culture with V19S76K-5C8 than with medium control (FIG. 16), indicating V19S76K-5C8-induced lysis of CLL cells.

### Conclusion

The bispecific anti-CD40-anti-Vγ9Vδ2 TCR VHH V19S76K-5C8 induces cytotoxicity of CLL cells by autologous Vγ9Vδ2-T cells.

### Example 18: Bispecific VHH is Active Against Primary Multiple Myeloma

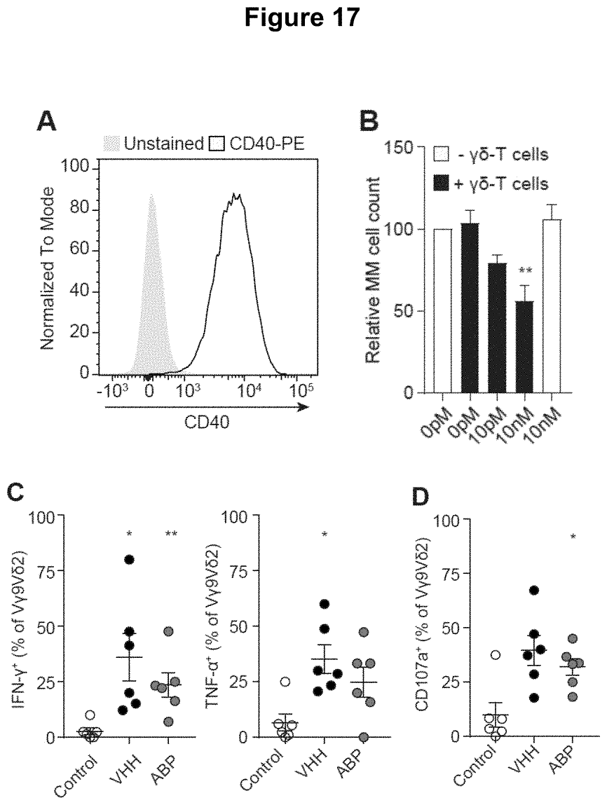

Because CD40 is also expressed on primary multiple myeloma (MM) cells (Pellat-Deceunynck et al. (1994) Blood 84:2597) (FIG. 17A) and CD40 stimulation exerts various biological effects, including proliferation of MM cells, we assessed the efficacy of V19S76K-5C8 in primary bone marrow samples from MM patients. When cultured overnight in the presence of the bispecific VHH, healthy donor-derived Vγ9Vδ2-T cells lysed primary MM cells (FIG. 17B).

Furthermore, Vγ9Vδ2-T cells present in the bone marrow of these patients were triggered to produce the pro-inflammatory cytokines IFN-γ and TNF-α upon culture with V19S76K-5C8 (FIG. 17C). Similarly, Vγ9Vδ2-T cells present in bone marrow mononuclear cells from MM patients degranulated after culture with the bispecific VHH V19S76K-5C8 (FIG. 17D).

Together, these results indicate that V19S76K-5C8 is active against primary MM and can activate autologous bone marrow-derived Vγ9Vδ2-T cells.

### Example 19: Bispecific VHH Prevents Tumor Outgrowth in a Xenograft Model

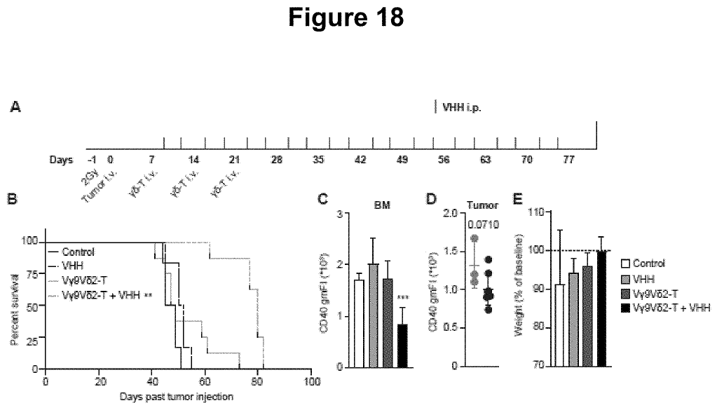

To study the effects of the bispecific VHH on tumor growth in vivo, immunodeficient NSG mice were injected with cells of MM.1s, a human multiple myeloma cell line. The tumor cells were allowed to grow out and engraft for 1 week before mice received the first of three weekly i.v. injections with either human Vγ9Vδ2-T cells or PBS, followed by twice weekly i.p. injections with V19S76K-5C8 or PBS (FIG. 18A). Neither V19S76K-5C8 alone or the Vγ9Vδ2-T cells alone significantly improved overall survival. In contrast, mice treated with both V19S76K-5C8 and Vγ9Vδ2-T cells lived significantly longer, with a median overall survival of 80 days versus 47 days in the control group (FIG. 18B).

At the time of sacrifice, CD40 expression was significantly lower on malignant cells in the bone marrow of mice treated with both V19S76K-5C8 and Vγ9Vδ2-T cells than of control mice (FIG. 18C). A similar trend was observed for malignant plasma cells in macroscopically identified plasmacytomas (FIG. 18D).

Mice treated with both V19S76K-5C8 and Vγ9Vδ2-T cells retained their initial body weight after 7 weeks of treatment (FIG. 18E).

In conclusion, the bispecific VHH improves survival in a MM in vivo model in a Vγ9Vδ2-T cell-dependent manner.

1. A multispecific antibody comprising a first antigen-binding region capable of binding human CD40 and a second antigen-binding region capable of binding a human Vγ9Vδ2 T cell receptor.

2. The multispecific antibody according to claim 1, wherein the multispecific antibody is a bispecific antibody.

3. The multispecific antibody according to any one of the preceding claims, wherein the first antigen-binding region is a single-domain antibody.

4. The multispecific antibody according to any one of the preceding claims, wherein the second antigen-binding region is a single-domain antibody.

5. The multispecific antibody according to any one of the preceding claims, wherein the first antigen-binding region and second antigen-binding region are covalently linked via a peptide linker.

6. The multispecific antibody according to claim 5, wherein the peptide linker comprises or consists of the sequence set forth in SEQ ID NO:21.

7. The multispecific antibody according to any one of the preceding claims, wherein the first antigen-binding region is located N-terminally of the second antigen-binding region.

8. The multispecific antibody according to any one of the preceding claims, wherein the multispecific antibody binds monovalently to CD40 and binds monovalently to the human Vγ9Vδ2 T cell receptor.

9. The multispecific antibody according to any one of the preceding claims, wherein the multispecific antibody is not an agonist of human CD40.

10. The multispecific antibody according to any one of the preceding claims, wherein the multispecific antibody is an antagonist of human CD40.

11. The multispecific antibody according to any one of the preceding claims, wherein the multispecific antibody is capable of sensitizing human CD40-expressing cells to venetoclax.

12. The multispecific antibody according to any one of the preceding claims, wherein the multispecific antibody competes for binding to human CD40 with an antibody having the sequence set forth in SEQ ID NO:13 and/or competes for binding to human CD40 with an antibody having the sequence set forth in SEQ ID NO: 14.

13. The multispecific antibody according to any one of the preceding claims, wherein the multispecific antibody binds the same epitope on human CD40 as an antibody having the sequence set forth in SEQ ID NO:13 or binds the same epitope on human CD40 as antibody having the sequence set forth in SEQ ID NO: 14.

14. The multispecific antibody according to any one of the preceding claims, wherein the first antigen-binding region comprises
the VH CDR1 sequence set forth in SEQ ID NO:1, the VH CDR2 sequence set forth in SEQ ID NO:2 and the VH CDR3 sequence set forth in SEQ ID NO:3, or
the VH CDR1 sequence set forth in SEQ ID NO:4, the VH CDR2 sequence set forth in SEQ ID NO:5 and the VH CDR3 sequence set forth in SEQ ID NO:6.

15. The multispecific antibody according to any one of the preceding claims, wherein the first antigen-binding region is humanized.

16. The multispecific antibody according to any one of the preceding claims, wherein the first antigen-binding region comprises or consists of:
the sequence set forth in SEQ ID NO:13 or the sequence set forth in SEQ ID NO:14, or
a sequence having at least 90%, such as least 92%, e.g. at least 94%, such as at least 96%, e.g. at least 98% sequence identity to the sequence set forth in SEQ ID NO:13 or a sequence having at least 90%, such as least 92%, e.g. at least 94%, such as at least 96%, e.g. at least 98% sequence identity to the sequence set forth in SEQ ID NO: 14.

17. The multispecific antibody according to any one of the preceding claims, wherein the multispecific antibody is able to activate human Vγ9Vδ2 T cells.

18. The multispecific antibody according to any one of the preceding claims, wherein the multispecific antibody is capable of mediating killing of human CD40-expressing cells from a chronic lymphocytic leukemia patient and/or from a multiple myeloma patient.

19. The multispecific antibody according to any one of the preceding claims, wherein the multispecific antibody is capable of mediating killing of human CD40-expressing cells from a chronic lymphocytic leukemia patient that have been stimulated with CD40L.

20. The multispecific antibody according to any one of the preceding claims, wherein the multispecific antibody is capable of binding to human Vδ2.

21. The multispecific antibody according to any one of the preceding claims, wherein the multispecific antibody competes for binding to human Vδ2 with an antibody having the sequence set forth in SEQ ID NO: 17 or competes for binding to human Vδ2 with an antibody having the sequence set forth in SEQ ID NO: 18.

22. The multispecific antibody according to any one of the preceding claims, wherein the multispecific antibody binds the same epitope on human Vδ2 as an antibody having the sequence set forth in SEQ ID NO: 17 or binds the same epitope on human Vδ2 as an antibody having the sequence set forth in SEQ ID NO: 18.

23. The multispecific antibody according to any one of the preceding claims, wherein the second antigen-binding region comprises the VH CDR1 sequence set forth in SEQ ID NO:7, the VH CDR2 sequence set forth in SEQ ID NO:8 and the VH CDR3 sequence set forth in SEQ ID NO:9 or comprises the VH CDR1 sequence set forth in SEQ ID NO:10, the VH CDR2 sequence set forth in SEQ ID NO:11 and the VH CDR3 sequence set forth in SEQ ID NO: 12.

24. The multispecific antibody according to any one of the preceding claims, wherein the second antigen-binding region is humanized.

25. The multispecific antibody according to any one of the preceding claims, wherein the second antigen-binding region comprises or consists of
the sequence set forth in SEQ ID NO:17, or
a sequence having at least 90%, such as least 92%, e.g. at least 94%, such as at least 96%, e.g. at least 98% sequence identity to the sequence set forth in SEQ ID NO:17, or
a sequence selected from the group consisting of SEQ ID NO: 25, 26, 27, 28, 29, 30, 31, 32, 33 and 34.

26. The multispecific antibody according to any one of the preceding claims, wherein the first antigen-binding region comprises
the VH CDR1 sequence set forth in SEQ ID NO:1, the VH CDR2 sequence set forth in SEQ ID NO:2 and the VH CDR3 sequence set forth in SEQ ID NO:3, or
the VH CDR1 sequence set forth in SEQ ID NO:4, the VH CDR2 sequence set forth in SEQ ID NO:5 and the VH CDR3 sequence set forth in SEQ ID NO:6,
and wherein the second antigen-binding region comprises the VH CDR1 sequence set forth in SEQ ID NO:7, the VH CDR2 sequence set forth in SEQ ID NO:8 and the VH CDR3 sequence set forth in SEQ ID NO:9.

27. An antibody comprising a first antigen-binding region capable of binding human CD40, wherein the antibody competes for binding to human CD40 with an antibody having the sequence set forth in SEQ ID NO:13 and/or competes for binding to human CD40 with an antibody having the sequence set forth in SEQ ID NO: 14.

28. The antibody according to claim 27, wherein the antibody binds the same epitope on human CD40 as an antibody having the sequence set forth in SEQ ID NO:13 or binds the same epitope on human CD40 as antibody having the sequence set forth in SEQ ID NO: 14.

29. The antibody according to claim 27 or 28, wherein the first antigen-binding region comprises
the VH CDR1 sequence set forth in SEQ ID NO:1, the VH CDR2 sequence set forth in SEQ ID NO:2 and the VH CDR3 sequence set forth in SEQ ID NO:3, or
the VH CDR1 sequence set forth in SEQ ID NO:4, the VH CDR2 sequence set forth in SEQ ID NO:5 and the VH CDR3 sequence set forth in SEQ ID NO:6.

30. The antibody according to any one of claims 27 to 29, wherein the first antigen-binding region comprises or consists of:
the sequence set forth in SEQ ID NO:13 or the sequence set forth in SEQ ID NO:14, or
a sequence having at least 90%, such as least 92%, e.g. at least 94%, such as at least 96%, e.g. at least 98% sequence identity to the sequence set forth in SEQ ID NO:13 or a sequence having at least 90%, such as least 92%, e.g. at least 94%, such as at least 96%, e.g. at least 98% sequence identity to the sequence set forth in SEQ ID NO: 14.

31. The antibody according to any one of claims 27 to 30, wherein the first antigen-binding region is a single-domain antibody.

32. The antibody according to any one of claims 27 to 31, wherein the antibody is a monospecific antibody, e.g. a monovalent antibody.

33. The antibody according to any one of claims 27 to 31, wherein the antibody comprises a second antigen-binding region which binds an antigen which is not human CD40 or Vδ2.

34. The antibody according to any one of claims 27 to 33, having one or more of the properties defined in claims 9 to 11.

35. A pharmaceutical composition comprising a multispecific antibody according to any one of claims 1 to 26 or an antibody according to any one of claims 27 to 34 and a pharmaceutically-acceptable excipient.

36. The multispecific antibody according to any one of claims 1 to 26 or the antibody according to any one of claims 27 to 34 for use as a medicament.

37. The multispecific antibody according to any one of claims 1 to 26 or the antibody according to any one of claims 27 to 34 for use in the treatment of cancer.

38. The multispecific antibody according to any one of claims 1 to 26 or the antibody according to any one of claims 27 to 34 for use in the treatment of chronic lymphocytic leukemia, multiple myeloma, non-Hodgkin's lymphoma, Hodgkin's lymphoma, follicular lymphoma, head and neck cancer, pancreatic cancer, ovarian cancer, lung cancer, breast cancer, colon cancer, prostate cancer, B-cell lymphoma/leukemia, Burkitt lymphoma or B acute lymphoblastic leukemia.

39. The multispecific antibody according to any one of claims 1 to 26 for use in the treatment of chronic lymphocytic leukemia or multiple myeloma.

40. The multispecific antibody according to any one of claims 1 to 26 or the antibody according to any one of claims 27 to 34 for use according to any one of claims 36 to 39, wherein the use is in combination with a Bcl-2 blocker, such as venetoclax.

41. A method of treating a disease comprising administration of a multispecific antibody according to any one of claims 1 to 26 or an antibody according to any one of claims 27 to 34 to a human subject in need thereof.

42. The method according to claim 41, wherein the disease is cancer.

43. A nucleic acid construct encoding the multispecific antibody according to any one of claims 1 to 26 or the antibody according to any one of claims 27 to 34.

44. An expression vector comprising a nucleic acid construct according to claim 43.

45. A host cell comprising a nucleic acid construct according to claim 43 or an expression vector according to claim 44.
